(19) United States
(12) Patent Application Publication
(10) Pub. No.: US 2022/0251025 A1
(43) Pub. Date: Aug. 11, 2022

(54) AUTOMATED ULTRA-COMPACT MICRODROPLET RADIOSYNTHESIZER

(71) Applicant: THE REGENTS OF THE UNIVERSITY OF CALIFORNIA, Oakland, CA (US)

(72) Inventors: R. Michael van Dam, Sherman Oaks, CA (US); Jia Wang, Los Angeles, CA (US)

(73) Assignee: THE REGENTS OF THE UNIVERSITY OF CALIFORNIA, Oakland, CA (US)

(21) Appl. No.: 17/612,206

(22) PCT Filed: May 22, 2020
(86) PCT No.: PCT/US2020/034336

§ 371 Date: Nov. 17, 2021

Related U.S. Application Data
(60) Provisional application No. 62/851,207, filed on May 22, 2019.

(51) Int. Cl. C07C 227/16; B01L 9/00; C07B 59/00; B01J 19/00; B01L 3/00

(52) U.S. Cl. CPC C07C 227/16; B01L 9/527; C07B 59/001; B01J 19/0093; C07B 2200/05; B01L 2200/025; B01L 2300/1822; B01L 2300/1844; B01L 2400/049; B01L 3/502715

(57) ABSTRACT

A chemical synthesis platform based on a particularly simple chip is described herein, where reactions take place atop a hydrophobic substrate patterned with a circular hydrophilic liquid trap. The overall supporting hardware (heater, rotating carousel of reagent dispensers, etc.) can be packaged into a very compact format (about the size of a coffee cup). We demonstrate the consistent synthesis of [¹⁸F]fallypride with high yield, and show that protocols optimized using a high-throughput optimization platform we have developed can be readily translated to this device with no changes or reoptimization.

### RELATED APPLICATION

This application claims priority to U.S. Provisional Patent Application No. 62/851,207 filed on May 22, 2019, which is hereby incorporated by reference in its entirety. Priority is claimed pursuant to 35 U.S.C. § 119 and any other applicable statute.

### STATEMENT REGARDING FEDERALLY SPONSORED RESEARCH AND DEVELOPMENT

This invention was made with government support under Grant Numbers AG049918, CA212718, and MH097271, awarded by the National Institutes of Health. The government has certain rights in the invention.

### TECHNICAL FIELD

The technical field generally relates to devices used for radiosynthesis. In particular, the technical field relates to an automated yet compact radiosynthesizer device using droplet processes.

### BACKGROUND

Positron emission tomography (PET) is a non-invasive medical imaging method that can be used as a research tool for studying the biological processes involved in the course of diseases and making critical measurements during the development of new drugs. It is also widely used in the clinic to diagnose and stage disease, predict treatment response, and evaluate efficacy of treatment; furthermore, PET can also be used to help guide treatment and serves a critical role in the emerging field of personalized medicine. Shortly before undergoing a PET imaging procedure, the patient (or subject) must be injected with a short-lived radiolabeled compound (e.g., a tracer), which is designed to highlight a particular biological target or pathway.

The current processes and technologies for producing these PET “tracers” are complex and expensive, which greatly hinders research efforts into the development and validation of novel tracers, or the translation of new tracers into the clinic. For more than a decade, investigators have been exploring the use of microfluidics to improve the production of PET tracers and have advanced this technology to the point of demonstrating production of tracers suitable for clinical use (see, e.g., M.-Q. Zheng, L. Collier, F. Bois, O. J. Kelada, K. Hammond, J. Ropchan, M. R. Akula, D. J. Carlson, G. W. Kabalka and Y. Huang, Nucl. Med. Biol., 2015, 42, 578-584; S. H. Liang, D. L. Yokell, M. D. Normandin, P. A. Rice, R. N. Jackson, T. M. Shoup, T. J. Brady, G. El Fakhri, T. L. Collier and N. Vasdev, Mol. Imaging, 2014, 13, 1-5; A. Lebedev, R. Miraghaie, K. Kotta, C. E. Ball, J. Zhang, M. S. Buchsbaum, H. C. Kolb and A. Elizarov, Lab. Chip, 2012, 13, 136-145). All references cited herein are hereby incorporated by reference in their entirety, and for all purposes.

These studies, especially the use of micro-volume reactors or droplet-based reactors, have revealed several important advantages of microfluidics in radiochemistry that can reduce the cost and complexity of PET tracer production. Though all uses of PET tracers can benefit, the improvements will be especially impactful for the small batches needed in research applications or in the initial studies to develop novel tracers and translate them to the clinic. Particularly important advantages of small-volume radiosynthesizers compared to conventional synthesizers are the significant reduction in footprint of the radiochemistry setup, enabling self-shielding rather than requiring operation within specialized “hot cells”, and the 2-3 orders of magnitude reduction in consumption of expensive reagents (e.g., precursors, peptides, etc.). Microvolume synthesis has also been shown to boost the molar activity of tracers produced via isotope exchange and can achieve high molar activities even when producing small batches of tracers, both of which are not possible in conventional systems unless very high amounts of radioactivity are used.

As a testament to the versatility of droplet-based approaches, a wide range of PET tracers have been synthesized using these methods, including [F]fallypride, [¹⁸F]FDG, [¹⁸F]FLT, [¹⁸F]SFB, [¹⁸F]FDOPA, sulfonyl [¹⁸F]fluoride, [¹⁸F]FMISO, [¹⁸F]FES, [¹⁸F]AMBF₃-TATE, etc. In addition, these microscale reactors are scalable, with the possibility to produce clinically-relevant doses by increasing the concentration of radioisotope supplied into the system.

Droplet-radiochemistry platforms include electrowetting-on-dielectric (EWOD) devices and a more recent system using patterned wettability for passive droplet transport, due to the extremely small reaction volumes and straightforward fluidic system (see, e.g., J. Wang, P. H. Chao, S. Hanet and R. M. van Dam, Lab. Chip, 2017, 17, 4342-4355). In the passive transport approach, the chip consists of a Teflon® coated silicon wafer with patterned circular hydrophilic reaction zone in the center and several radial tapered channels to transport droplets from reagent loading sites at the periphery into the reaction zone. Though this approach significantly decreased the chip cost and complexity and could be used to successfully synthesize [¹⁸F]fallypride and [¹⁸F]FDG, it was found that the behavior of the droplets was sensitive to the solvent type, temperature, and volume, sometimes leading to unwanted spreading out of the solution along the tapered reagent pathways of the chip. Such spreading can adversely affect synthesis performance and lead to inconsistent results, requiring expenditure of time and effort to optimize reagents and solvents, loading protocols (timing and volumes) and other aspects to achieve high synthesis performance.

### SUMMARY

The present disclosure is directed to devices, systems and methods for performing radiosynthesis which avoid existing issues with current droplet-based approaches and which further streamline the adoption of new protocols to the microdroplet format. An even simpler microfluidic chip than previously used for radiosynthesis is disclosed herein which utilizes a simple reaction site, such as a circular hydrophilic reaction site or zone. Instead of reagents moving from multiple fixed loading sites on the chip (with each loading site located under a respective reagent dispenser) to the reaction zone spontaneously, the presently disclosed radiosynthesis device is designed to rotate the microfluidic chip under a carousel of reagent dispensers for on-demand loading of desired reagents when needed. This change was found to significantly improve the performance of the on-chip reaction, and the amount of the reaction product that could be collected from the chip. The system can be made very compact (e.g., similar to the size of a 12 ounce coffee cup), demonstrating that sophisticated multi-step radiochemistry can be accomplished with a very small apparatus. The compact size (10×6×12 cm; W×D×H, i.e., a volume of about 720 cm³), which includes the reagent handling system, microreactor, and temperature control system, is a tremendous advantage in radiochemistry facilities where shielded space is at a premium. For example, multiple droplet synthesizers could be operated inside a single hot cell, or the droplet synthesizer could be operated outside the hot cell by adding localized shielding surrounding the synthesizer.

Hence, in one embodiment of the present disclosure, a radiosynthesis device comprises a thermally controlled support configured to hold a microfluidic chip. The microfluidic chip has one or more reaction sites formed thereon. Each reaction site may be a circular-shaped hydrophilic region, or other suitable shape, such as a square, rectangle, etc. The radiosynthesis device has a fixture configured to hold a plurality of dispensers and a collection tube. A plurality of non-contact dispensers are installed on the fixture above the support and the microfluidic chip held in the support. Each non-contact dispenser is configured to respectively dispense one or more droplets of a respective reagent into the one or more reaction sites. A collection tube is also installed on the fixture above the support and the microfluidic chip held in the support. A motorized rotation stage is operably coupled to the support for controllably rotating the support and the microfluidic chip held in the support relative to the plurality of non-contact dispensers and the collection tube. For example, rotation of the microfluidic chip rotates the one or more reaction sites along an arc of a circle to the various dispensers and the collection tube. By rotating the microfluidic chip, the motorized rotation stage sequentially positions the one or more reaction sites at the non-contact dispensers for dispensing respective reagent for the particular synthesis being performed in each reaction site from the non-contact dispensers into the one or more reaction sites. For instance, a first reaction site may have reagent dispensed from a first dispenser, a second dispenser, a third dispenser, and a sixth dispenser, while a second reaction site may have reagent dispensed from a fourth dispenser, a fifth dispenser and the sixth dispenser. Then, the motorized stage sequentially positions the one or more reaction sites at the collection tube for removing reaction product from the one or more reaction sites via the collection tube.

In another aspect, the radiosynthesis device may further include a computing device for controlling the operation of the radiosynthesis device. The computing device has a software program executed on the computing device. The computing device may be any suitable personal computer, or other computer, such as a tablet computer, handheld computer, smartphone, or the like. For example, LabView, from National Instruments, may be used as the software program executing on a personal computer. The software program is configured to program the computer to control the temperature of the thermally controlled support, the operation of the motorized rotation stage, the dispensing of reagents by the non-contact dispensers, removal of reaction product by the collection tube, and/or other functions of the radiosynthesis device.

In still another aspect of the radiosynthesis device, the thermally controlled support may include a heater and a thermoelectric cooler in thermal contact with the microfluidic chip. In another feature, the radiosynthesis device also includes a heat sink in thermal contact with one or more of the heater and thermoelectric cooler.

In yet another aspect, the radiosynthesis device may also have a fan coupled to the fixture for circulating air over the heat sink.

In still another aspect, the radiosynthesis device also includes a collection vial fluidically coupled to the collection tube, and respective reagent tubes fluidically coupled to the plurality of non-contact dispensers and to respective reagent containers coupled to the fixture.

In another aspect of the radiosynthesis device, the microfluidic chip includes a plurality of hydrophilic reaction sites formed thereon and the reaction sites are disposed along an arc on the surface of the microfluidic chip.

In still another aspect, the radiosynthesis device further includes a data acquisition device which interfaces the computing device and the components of the radiosynthesis device, such as the temperature controlled support, the motorized rotation stage, the non-contact dispensers, and a vacuum regulator and/or vacuum source which control the withdrawal of reaction product via the collection tube.

In another feature of the radiosynthesis device, the motorized rotation stage and fixture and/or other components of the device may be mounted within a housing. The housing may be a gas-tight enclosure which contains any emitted solvent vapor or radioactive vapor from escaping, may also include radiation shielding, for example, of sufficient thickness for the intended synthesis/radioactivity.

In still another aspect, the radiosynthesis device may be a compact size, such as less than about 750 cm³, or having dimensions of no more than 10 cm×6 cm×12 cm (width×depth×height).

In still another aspect, the plurality of non-contact dispensers and the collection tube may be disposed in a cartridge or kit that is removably mounted on the fixture. For example, different cartridges may be specifically configured for different radiosynthesis protocols and collection configurations which can be quickly and easily swapped in and out of the radiosynthesis device. The cartridge or kit may be disposable or reusable. The cartridge or kit may also include pre-loaded reagents in the dispensers or in containers in fluid communication with the dispensers. The cartridge or kit may include one or more of: reagent containers for a specific synthesis; reagent tubing between the reagent containers and the dispensers; and/or the non-contact dispensers. For instance, the cartridge or kit may include just the reagent containers such that the reagent cartridge or kit is installed in the radiosynthesis device as a unitary module. In such case, a cleaning cassette or cartridge may be installed with cleaning reagents to perform a cleaning of the dispensers and tubing. Then, another cartridge or kit (for the same or a different synthesis) can be installed on the radiosynthesis device without contamination from the previous synthesis. Alternatively, the cartridge or kit may include the reagent containers, non-contact dispensers, and tubing therebetween. In this way, the entire cartridge or kit can be exchanged for different syntheses, without requiring a cleaning step, as all or most of the components which contact the reagents is within the cartridge or kit.

In another embodiment, the radiosynthesis device is configured to utilize a single microfluidic chip having multiple reaction sites disposed along an arc of a circle which can be rotated to each of the reagent dispensers and collection tube. This advantageously would allow multiple reactions to be run in parallel. This could also be useful for synthesizing a PET tracer at several different conditions (reagent concentrations, etc.) to perform high-throughput optimization of the radiosynthesis process in a very compact space and short time. Multiple reaction locations also enable synthesizing multiple batches of the same tracer. It is possible that when synthesizing high radioactivity batches of a tracer, the yield may suffer due to radiolysis. Splitting the synthesis into multiple droplets can mitigate the effects, and then the product in each of the reaction sites can be pooled at the end resulting in a larger-scale batch. Finally, different tracers could be synthesized in a back-to-back fashion (if sufficient reagent dispensers are available). By slightly increasing the radius of the arc, additional reagent dispensers (and reaction sites) may be added.

In one embodiment, the radiosynthesis device is used in conjunction with microfluidic chips containing multiple reaction sites arranged in a circular pattern centered on the axis of rotation. The multiple reaction sites can be used to perform optimization studies, to assist with scale-up of activity level, or to synthesize multiple different radiolabeled compounds. The advantage of such approaches is increased throughput and/or increased safety (i.e., avoid handling the chip, which has some residual radioactivity, between syntheses). For applications that may need a large number of reagents, the dispensers may be cleaned between reactions, and a new set of reagent containers/vials installed for the dispensers.

For optimization studies, each reaction site can be an individual experiment to explore the impact of different reaction conditions on the synthesis performance (e.g., yield). A wide range of reaction conditions can be explored in this fashion, including concentrations, reaction solvent, reaction time, and reaction temperature. (a) For concentration or solvent studies, all reaction sites can be loaded initially, and then all reactions performed simultaneously by heating the whole chip for the desired reaction time at the desired temperature. After reaction, the reaction products can be collected sequentially (via the collection tubing) for analysis. Cleaning of the collection tubing can be performed by dispensing cleaning solution to the just-collected reaction site (or a blank site) and flushing it through the collection tubing. The different reagent concentrations (or reagents in different solvents) can be prepared in advance and dispensed via dedicated dispensers, or the different reagent concentrations can be prepared on the fly by dispensing different combinations of a concentration reagent stock solution from one dispenser and a dilution solution from another dispenser. (b) For temperature and time studies, reactions can be performed in sequence: first, reagents would be loaded to one reaction site, and the chip would then be heated to the desired temperature for the desired time and the product collected from the chip. After the chip is cooled, reagents can be loaded to the next reaction site, and the chip can be heated to the desired temperature for the desired time and the product collected for analysis, etc.

Multiple reaction sites can also be used for scale-up studies. In some cases, it has been observed that increasing the amount of radioactivity in the reaction (which leads to a higher radioactivity concentration) can lead to radiolysis, i.e., degradation of the product due to radiation that is being emitted. Typically, it has been observed that there is a range of activity levels below which there is no adverse effect, and then a threshold level above which the synthesis performance starts to decline as radioactivity is further increased. To enable scale-up above this threshold, it is possible to divide up the amount of activity among multiple individual reactions (such that each one is below the threshold amount), perform the multiple reactions in parallel, and then pool the reaction products at the end. During this pooling phase, the radioactivity concentration does not increase and thus radiolysis does not occur. (Furthermore, at the end of the reaction, it is possible to add radiolysis stabilizers as a further protective measure; these stabilizers usually cannot be present during the reaction.)

Finally, the multiple reaction sites can also be used to make multiple different radiolabeled compounds in sequence. First, reagents are dispensed to one reaction site, the reaction is carried out, and the product collected for purification and formulation and ultimately imaging. Next, reagents are loaded to the second reaction site, the reaction performed, and the second product collected via the collection tubing for purification and formulation and ultimately imaging. Cleaning can be performed between compounds as described herein.

Another embodiment of the present disclosure is directed to a radiosynthesis system comprising the radiosynthesis device. In the radiosynthesis system, an upstream radionuclide concentrator (also referred to as a radioisotope concentrator) is connected to the radiosynthesis device upstream of the radiosynthesis device. The radionuclide concentrator is configured to concentrate a radioisotope and output the concentrated radioisotope to the radiosynthesis device. This increases the amount of radioactivity used in the synthesis process. In another aspect of the radiosynthesis system, a downstream purification and formulation module is connected to the radiosynthesis device downstream of the radiosynthesis device. For example, purification can be carried out using an analytical-scale HPLC system or cartridge purification.

Another embodiment of the present disclosure is directed to a method of using the radiosynthesis devices and systems disclosed herein. In one embodiment, the method includes dispensing one or more droplets of reagent onto the one or more reaction sites of the microfluidic chip using the plurality of non-contact dispensers, wherein the microfluidic chip is rotated into position under the respective non-contact dispensers by the motorized rotation stage. The one or more droplets of reagent are heated and/or cooled with the thermally controlled support. The microfluidic chip is rotated using the motorized rotation stage to place the one or more reaction sites containing a droplet under the collection tube. The reaction product in the reaction sites is removed with the collection tube by applying a vacuum to the collection tube. In another aspect, the synthesis may include one or more additional reaction steps prior to collection, each including: (i) evaporating solvent (optional); (ii) dispensing one or more droplets of reagent onto the one or more reaction sites (which also may include rotating the microfluidic chip to a non-contact dispenser using the motorized rotation stage; (iii) heating the reactants to a reaction temperature using the thermally controlled support; and cooling the reactants to a desired temperature prior to the next step of the synthesis.

Another embodiment of the present disclosure is directed to a method of using the radiosynthesis device to produce a radiochemical, such as a PET tracer. In this exemplary method, the synthesis includes two reaction steps, fluorination and deprotection. The syntheses of some PET tracers do not require a deprotection step, and some tracers have other non-deprotection reactions, instead of, or in addition to, the deprotection step. Accordingly, the exemplary method may be modified accordingly,

The method commences with dispensing one or more droplets of a radioisotope stock solution comprising a radioisotope in a solvent onto a first reaction site of the one or more reaction sites of the microfluidic chip using a first dispenser of the plurality of non-contact dispensers. The stock solution may include a base and phase transfer catalyst, which may be premixed into the stock solution, or introduced during upstream processing (e.g., by a radionuclide concentrator, or they can be dispensed into the reaction site (before or after the radioisotope stock solution is dispensed). Next, the radioisotope stock solution on the first reaction site is thermally treated (e.g., heating and/or cooling) using the thermally controlled support to evaporate the solvent leaving a dried residue of radioisotope complex on the first reaction site. Then, the microfluidic chip is rotated relative to the dispensers by rotating the motorized rotation stage to position the first reaction site at a second dispenser of the plurality of non-contact dispensers. One or more droplets of a precursor solution are dispensed onto the first reaction site using the second dispenser to dissolve the dried residue of radioisotope complex resulting in a solution of precursor solution and radioisotope complex. The microfluidic chip is rotated again by rotating the motorized rotation stage to position the first reaction site at a third dispenser of the plurality of non-contact dispensers. With the first reaction site positioned at the third dispenser, the solution of precursor solution and radioisotope complex on the first reaction site is thermally treated (e.g., heated and/or cooled) using the thermally controlled support to perform a fluorination reaction thereby producing a fluorinated reaction product. Optionally, during the fluorination reaction, a replenishing reagent may be dispensed periodically onto the first reaction site using the third dispenser during the fluorination reaction. Next, the microfluidic chip is rotated by rotating the motorized rotation stage to position the first reaction site at a fourth dispenser of the plurality of non-contact dispensers. The fourth dispenser dispenses one or more droplets of a deprotection solution onto the first reaction site containing the fluorinated reaction product. The deprotection solution and fluorinated reaction product on the first reaction site are thermally treated using the thermally controlled support to perform a deprotection reaction thereby producing crude radiochemical product. The microfluidic chip is rotated by rotating the motorized rotation stage to position the first reaction site at a fifth dispenser of the plurality of non-contact dispensers. The fifth dispenser dispenses one or more droplets of a collection solution onto the first reaction site containing crude radiochemical product to dilute the crude radiochemical product. The microfluidic chip is rotated by rotating the motorized rotation stage to position the first reaction site at the collection tube. Then, the diluted crude radiochemical product is removed from the first reaction site using the collection tube by applying a vacuum to the collection tube.

In another aspect of the method of synthesizing a radiochemical, the process of collecting the diluted crude radiochemical product from the first reaction site may include repeating the dilution and removal steps multiple times For instance, the following process may be repeated a suitable number of times: rotating the microfluidic chip by rotating the motorized rotation stage to position the first reaction site back to the fifth dispenser and dispensing one or more droplets of a collection solution onto the first reaction site containing crude radiochemical product; and rotating the microfluidic chip by rotating the motorized rotation stage to position the first reaction site at the collection tube and removing the diluted crude radiochemical product with the collection tube by applying a vacuum to the collection tube. For instance, this collection process may be repeated two, three, four, five, or more times.

In another aspect of the method of synthesizing a radiochemical, the diluted crude radiochemical may be conveyed through the collection tube to a collection vial using a vacuum source connected to the collection vial.

The method embodiments of using the radiosynthesis devices may include any one or more of the various aspects of the radiosynthesis device.

In order to confirm the advantages of the radiosynthesis device of the present disclosure compared to previous radiosynthesizer technologies (including conventional systems and microscale systems), a radiosynthesis device (also referred to as a “microdroplet reactor”) according to the disclosed embodiments was constructed. The microdroplet reactor includes three non-contact dispensers, including a [¹⁸F]fluoride/TBAHCO₃ dispenser (first dispenser), a precursor dispenser (second dispenser), a collection solution dispenser (third dispenser), and a collection tube, each sequentially positioned 90° counterclockwise from the preceding element along an arc about the rotational axis of the motorized rotation stage. The microfluidic chip for the microdroplet reactor was constructed with a single, circular hydrophilic reaction site. The microdroplet reactor was tested to synthesis a commonly used PET tracer, namely [¹⁸F]fallypride, for which radiosynthesis data was readily available for a number of previous radiosynthesizer technologies.

First, a mock synthesis of [¹⁸F]fallypride was performed on the microdroplet reactor to test the functionality. Then, the synthesis of [¹⁸F]fallypride was carried out using the microdroplet reactor to compare to the previous radiosynthesizer technologies. The synthesis of [¹⁸F]fallypride has a single reaction step, namely fluorination. In addition, syntheses of [¹⁸F]FET and [¹⁸F]FDOPA were carried out separately to test the versatility of the microdroplet reactor. The synthesis of [¹⁸F]FET and [¹⁸F]FDOPA have two reaction steps, namely, fluorination and deprotection, as described herein. Droplet-synthesis of other PET tracers can be carried out using the disclosures herein, with some variations and/or a reasonable amount of experimentation by those of ordinary skill in the art. Such droplet-syntheses may require any suitable number of reagent dispensers, such as two, three, four, five, six, or more dispensers.

During the mock synthesis, it was observed that the rotation stage moves the chip quickly and accurately to each desired position, the reagents were accurately delivered to the reaction sites without any visible splashing, and the solutions on the chip remained confined to the reaction site during all steps of the synthesis process.

To carry out the synthesis of [¹⁸F]fallypride on the microdroplet reactor, the microfluidic chip (also referred to as a “chip” for brevity) was first rotated by rotating the motorized rotation stage to position the reaction site below the [¹⁸F]fluoride/TBAHCO₃ dispenser (first dispenser) and eight 1 μL droplets of [¹⁸F]fluoride/TBAHCO₃ solution (˜8.9 MBq; ˜0.24 mCi) were sequentially loaded onto the chip (total time <10 seconds (s)). The chip was rotated 45° counterclockwise (CCW) and heated to 105° C. for 1 minute (“min”) to evaporate the solvent and leave a dried residue of the [¹⁸F]TBAF complex at the reaction site. Then, the chip was rotated 45° CCW to position the reaction site under the precursor dispenser (second dispenser) and twelve 0.5 μL droplets of precursor solution were loaded to dissolve the dried residue. Next, the chip was rotated 45° CCW and heated to 110° C. for 7 min to perform the radiofluorination reaction. Afterwards, the chip was rotated 45° CCW to position the reaction site under the collection solution dispenser (third dispenser), and twenty 1 μL droplets of collection solution were deposited to dilute the crude product. After rotating the chip 90° CCW to position the reaction site under the collection tube, the diluted solution was transferred into a collection vial by applying vacuum. The collection process was repeated a total of four times to minimize the residue on the chip (i.e., by rotating the chip 90° CW back to the collection solution dispenser, loading more collection solution, etc.).

Similar operations were carried out for the synthesis of [¹⁸F]FET and [¹⁸F]FDOPA on the microdroplet reactor. The crude radiochemical yields (RCYs) of [¹⁸F]fallypride, [¹⁸F]FET, and [¹⁸F]FDOPA were 96±3% (n=9), 70±9% (n=8) and 21±3% (n=3), respectively. These yields are significantly higher than when the droplet syntheses were manually performed (87±1% (n=6), 59±7% (n=4) and 18.8±0.2% (n=4), respectively). Additionally, even with 10's to 100's of times less precursor, the isolated RCY obtained after purification for all tracers were either significantly higher than or comparable to the macroscale syntheses, i.e., 78% (n=1) vs 66±8% (n=6) for [¹⁸F]fallypride, 64% (n=1) vs 55±5% (n=22) for [¹⁸F]FET, and 15.1±1.6% (n=3) vs 14±4% (n not reported) for [¹⁸F]FDOPA. The synthesis times (including purification and formulation) of all tracers using the microdroplet reactor were also much shorter (30 min for [¹⁸F]fallypride, 37 min for [¹⁸F]FET, and 40 min for [¹⁸F]FDOPA) than the time needed for macroscale syntheses (56 min (not including formulation) for [¹⁸F]fallypride, 63 min for [¹⁸F]FET, and 117 min for [¹⁸F]FDOPA).

FIG. 17 is a table showing a comparison of the presently disclosed microdroplet reactor and various previously disclosed radiosynthesizers (both microscale and macroscale) that have been used for the synthesis of [¹⁸F]fallypride. In the table of FIG. 17, total synthesis time includes purification and formulation. Total system size includes all hardware requiring shielding that is needed to perform the synthesis (not including purification and formulation). All RCY values are decay corrected. Where applicable, values are expressed as average±standard deviation, computed from the indicated number of measurements. N.A. indicates not available. N.S. indicates not specified. In the EWOD chip, droplet manipulation and temperature control are performed automatically, but loading of reagent droplets and collection of crude products are performed manually via pipette. In the PDMS reactor (Zhang et al. from Vanderbilt), fluid is automatically loaded into the chip via syringe pump, but manual activation of numerous components (switching valve states, opening evaporation vent in reactor, switching reagent connections, and hot plate heating) is needed. In flow-through reactions such as the Advion NanoTek, scaling up to higher activity levels will increase the amount of precursor consumed. To synthesize [¹⁸F]fallypride using the Advion NanoTek system, three different modules are needed, including a drying module, a syringe pump module and a capillary reactor module.

As illustrated in the comparison table of FIG. 17, the droplet-based radiosynthesis device of the present disclosure can quickly and efficiently synthesize the PET tracer [¹⁸F]fallypride, among other radiochemical products. As shown in FIG. 17, it can be seen that the microdroplet reactor enables the highest radiochemical yield (RCY), shortest synthesis time, and lowest amount of precursor compared to the various other systems used for the synthesis of this tracer. Furthermore, the present microdroplet reactor platform is able to leverage other efforts to develop high-throughput radiochemistry methods (i.e., using arrays of hydrophilic reaction zones on a single chip) (see, e.g., Rios, A., Wang, J., Chao, P. H., & van Dam, R. M. (2019). A novel multi-reaction microdroplet platform for rapid radiochemistry optimization. RSC Advances, 9(35), 20370-20374; A. Rios, J. Wang, P. H. Chao and R. M. Van Dam, in Proceedings of the 22nd International Conference on Miniaturized Systems for Chemistry and Life Sciences, Royal Society of Chemistry, Kaohsiung, Taiwan, 2018, pp. 1065-1067). Because the reaction site of the microdroplet reactor system and the droplet process is carried out in an identical fashion as on the high-throughput radiochemistry chips, the optimum protocol can be rapidly translated to the new automated platform with zero changes. As a result of this simplified approach, the radiosynthesis devices and methods of the present disclosure enable the low-cost production of diverse tracers for research as well as clinical applications.

The advantages of the microdroplet reactors and methods disclosed herein include the compact size of the overall microdroplet reactors. The apparatus (10×6×12 cm, W×D×H) of the microdroplet reactor is over an order of magnitude smaller than commercial macroscale synthesizers that are currently considered to be very compact (e.g., IBA RadioPharma Solutions Synthera® has dimensions 17×29×28.5 cm, W×D×H). Further, the apparatus is also much smaller than the commercial microfluidic-based radiosynthesizer NanoTek® from Advion (which includes a drying module, a syringe pump module and a capillary reactor module). The compact size of the presently disclosed microdroplet reactors also allow multiple microdroplet synthesizers to be operated in a single hot cell or mini-cell (a smaller type of hot cell). The microdroplet reactor(s) may also be operated without the specialized infrastructure of a radiochemistry lab. For one reason, the compact size of the microdroplet reactors requires much less shielding than a traditional macroscale radiosynthesizer. While the latter must be located in a hot cell weighing several tons, the microfluidic chip can be shielded with the same thickness walls of a hot cell and be light enough in weight to be used on the benchtop.

Moreover, the microscale radiochemical reactions of the present microdroplet reactors largely reduce the cost of reagents. Using microliter scale reactions, <1% of the amount of reagents used for macroscale reactions are needed while maintaining similar or higher concentrations. Thus, this enables significant reduction in cost of preparing radiopharmaceuticals.

Furthermore, the synthesis times using the present microdroplet reactors are typically 50% less than conventional (macroscale) technologies. This improves the overall yield by reducing radioactive decay of the radiochemical product. In addition, the radiosynthesis devices described herein achieve high radioactivity recovery. Due to the simple and direct design of the microfluidic chip and collection system, less than 1% radioactivity is left as residue on the chip and the collection tube, and the radioactivity recovery is much higher compared to passive transport-based chips. Fast and easy purification is also possible. Due to the small amount of reagents (i.e., base, precursor) used in the microdroplet reactions of the present radiosynthesis devices, the crude product can be purified via analytical-scale HPLC as compared to the semi-preparative HPLC used in conventional radiosynthesis. This results in short retention times (and short purification times) and lower mobile phase volume of the collected pure fraction (simplifying and shortening the formulation process).

The present radiosynthesis devices also offer easy adaption of the protocol optimized on the high-throughput microfluidic chip. Currently, for new PET tracers explored in microscale synthesis, one routinely performs an initial optimization process where dozens of reactions are manually performed under different conditions to determine the optimal reaction parameters. These studies are currently performed using a multi-reaction high-throughput radiochemistry chip. Because of the similar design of the reaction sites on those high-throughput chips and the microfluidic chip used in the present radiosynthesis devices (e.g., both utilize a silicon chip having a hydrophobic, circular reaction site), the protocol optimized on the high-throughput chip can be directly translated to the radiosynthesis devices disclosed herein in order to provide automated synthesis without further re-optimization.

### BRIEF DESCRIPTION OF THE DRAWINGS

FIG. 1A illustrates a microfluidic chip having a single hydrophilic reaction site, for use with a radiosynthesis device of FIG. 2A, according to one embodiment. The scale bar is 4 mm, and the diameter of the hydrophilic reaction site is 4 mm.

FIG. 1B schematically illustrates a photolithography process for fabrication of the microfluidic chip of FIG. 1A, according to one embodiment.

FIG. 2A is a side, perspective view of a solid model of a radiosynthesis device, according to one embodiment, alongside a 12 oz. coffee cup showing an exemplary scale of the radiosynthesis device, according to one embodiment.

FIG. 2B is photograph of an exemplary radiosynthesis device constructed substantially according to the drawing of FIG. 2A;

FIG. 2C is an enlarged, side, perspective view of the radiosynthesis device illustrated in FIG. 2A.

FIG. 3 schematically illustrates a control system used in the radiosynthesizer device of FIG. 2A, according to one embodiment.

FIG. 4A is a top view schematic of the (rotatable) microfluidic chip and (fixed) locations of reagent dispensers and the collection tube. The angle marker shows the center of rotation of the microfluidic chip on the rotation stage of the radiosynthesis device of FIG. 1A.

FIG. 4B shows an exemplary synthesis process for synthesizing [¹⁸F]fallypride, according to one embodiment.

FIG. 4C illustrates a process of using the radiosynthesis device of FIG. 2A to synthesize [¹⁸F]fallypride, in which each step is depicted by a schematic of the orientation of the microfluidic chip relative to the dispensing region of the dispensers and a corresponding perspective view of the dispensers and microfluidic chip, according to one embodiment.

FIG. 5A illustrates an example of the activity distribution for an exemplary [¹⁸F]fallypride synthesis process performed on the radiosynthesis device of FIG. 2A and a microfluidic chip of FIG. 1A, visualized with Cerenkov luminescence imaging. Four example images are shown. The dashed circle marks the reaction site and the numerical value indicates the fraction of total residual activity on the chip that is present inside the reaction site.

FIG. 5B illustrates an example of the activity distribution for an exemplary [¹⁸F]fallypride synthesis process performed on a passive transport chip after collection of crude product, visualized with Cerenkov luminescence imaging. Four example images are shown. The dashed circle marks the reaction site and the numerical value indicates the fraction of total residual activity on the chip that is present inside the reaction site.

FIG. 6A illustrates an HPLC chromatograms of crude [¹⁸F]fallypride product for an exemplary [¹⁸F]fallypride synthesis process performed on the radiosynthesis device of FIG. 2A and a microfluidic chip of FIG. 1A.

FIG. 6B illustrates an HPLC chromatogram of purified [¹⁸F]fallypride product for the exemplary [¹⁸F]fallypride synthesis process of FIG. 6A.

FIG. 6C illustrates an HPLC chromatogram of purified [¹⁸F]fallypride co-injected with fallypride reference standard for identity verification for the exemplary [¹⁸F]fallypride synthesis process of FIG. 6A. Radiochemical purity was 100%.

FIGS. 7A and 7B illustrate a comparison of Cerenkov images of developed radio-TLC plates spotted with crude [¹⁸F]fallypride product, in which FIG. 7A shows [¹⁸F]fallypride product synthesized on the radiosynthesis device of FIG. 2A, and FIG. 7B shows [¹⁸F]fallypride product synthesized on a passive transport chip.

FIGS. 8A and 8B illustrate a comparison of activity distribution on microfluidic chips of a radiosynthesis device of FIG. 2A after the collection step, visualized with Cerenkov luminescence imaging, in which FIG. 8A shows the activity distribution wherein a collection solution (80% MeOH/20% DI water, v/v) was dispensed on the reaction site of the microfluidic chip at 10 psi, and FIG. 8B shows the activity distribution wherein a collection solution (80% MeOH/20% DI water, v/v) was dispensed on the reaction site of the microfluidic chip at 5 psi. The dashed circle in FIGS. 8A and 8B shows the outline of the respective reaction site. The percentage ratio of residual activity at the reaction site to total residual activity on the entire microfluidic chip is indicated in the images.

FIG. 9A is a photographic image of a microfluidic chip having four hydrophilic reaction sites (e.g., for synthesis of [¹⁸F]FDOPA or other radiopharmaceutical), used in the examples described herein. The scale bar is 4 mm, the diameter of the each reaction site is 4 mm, and the pitch (center-to-center) between adjacent reaction sites is 9 mm.

FIG. 9B is a photographic image of a microfluidic chip having one hydrophilic reaction site (e.g., for synthesis of [¹⁸F]FDOPA or other radiopharmaceutical), used in the exampled described herein. The scale bar is 4 mm, and the diameter of the reaction site is 4 mm.

FIG. 10A schematically illustrates a synthesis scheme of [¹⁸F]FDOPA, according to one embodiment.

FIG. 10B schematically illustrates a manual [¹⁸F]FDOPA synthesis process using a multi-reaction chip.

FIGS. 11A-11C illustrate an optimization of a microdroplet synthesis of [¹⁸F]FDOPA using the manual synthesis process of FIG. 10B. FIG. 11A is a graph showing the effect of precursor concentration. FIG. 11B is a graph showing the effect of TEMPO concentration. FIG. 11C is a graph showing the effect of base amount, represented by K222 amount, which is 2.05 times the K₂CO₃ amount. The data points on the graphs represent average values and error bars represent standard deviations. For the 70 and 90 mol % datapoints in FIG. 10B, n=1, and the rest of the datapoints have n=2. For the datapoints in FIG. 10C, n=2.

FIG. 12A shows a top view schematic of a microfluidic chip mounted on the rotating stage and heating platform of the radiosynthesis device of FIG. 2A, and the fixed locations of the reagent dispensers and the collection tube above the microfluidic chip, according to one embodiment.

FIG. 12B illustrates a top view schematic of an automated [¹⁸F]FDOPA synthesis process using the radiosynthesis device of FIG. 2A, according to one embodiment.

FIGS. 13A-13C illustrate an optimization of reaction temperature for a synthesis process using the radiosynthesis device of FIG. 2A. FIG. 13A is a graph showing the effect of reaction temperature on the fluorination yield. FIG. 13B is a graph showing the effect of reaction temperature on the radioactivity recovery. FIG. 13C is a graph showing the effect of reaction temperature on the fluorination efficiency. The datapoints represent average values and error bars represent standard deviations. For 100, 105, 110, 120, 130, and 140° C. datapoints, the number of replicates is n=3, 2, 3, 3, 2, 2, respectively.

FIG. 14 illustrates a schematic of a [¹⁸F]FDOPA synthesis process when a cover plate is used during the deprotection step, according to one embodiment.

FIG. 15A is an example of a radio-HPLC chromatogram of crude [¹⁸F]FDOPA product, according to one example described herein.

FIG. 15B is an example of a radio-HPLC chromatograms of purified [¹⁸F]FDOPA product co-injected with a mixture of reference standards of both D-FDOPA and L-FDOPA, according to one example described herein.

FIG. 16 illustrates a schematic of a complete radiosynthesis system utilizing the radiosynthesis device of FIG. 2A, according to one embodiment.

FIG. 17 is a table showing a comparison of characteristics and performance of the presently disclosed radiosynthesis devices and various previously disclosed radiosynthesizers (both microscale and macroscale) that have been used for the synthesis of PET traces, such as [¹⁸F]fallypride.

### DETAILED DESCRIPTION OF ILLUSTRATED EMBODIMENTS

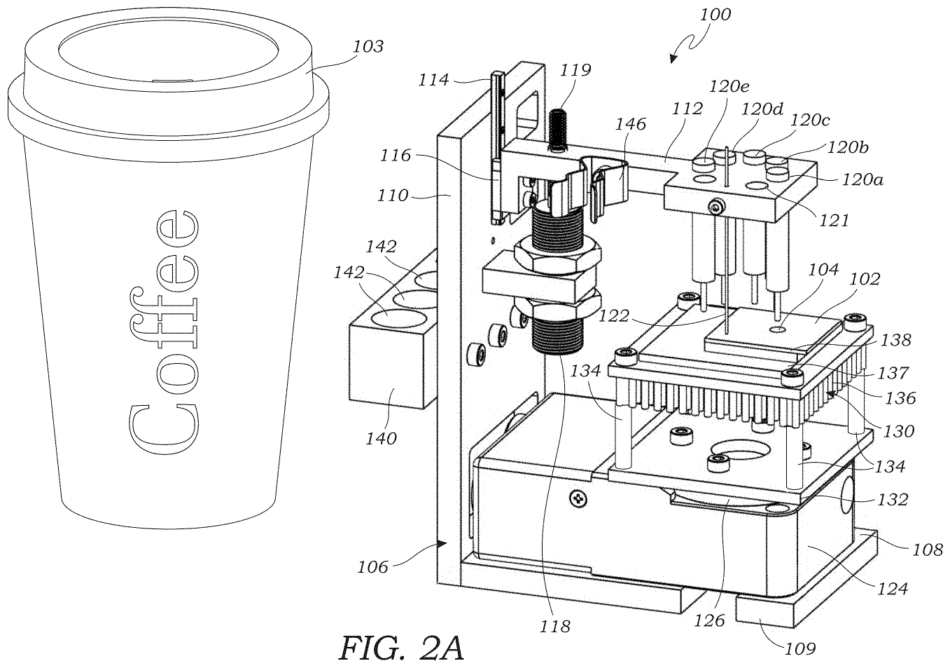

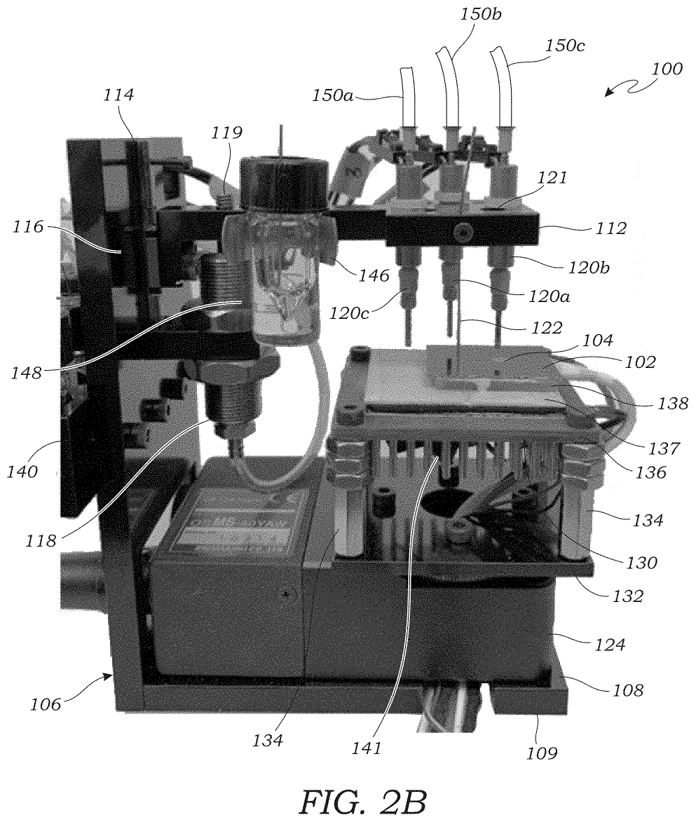

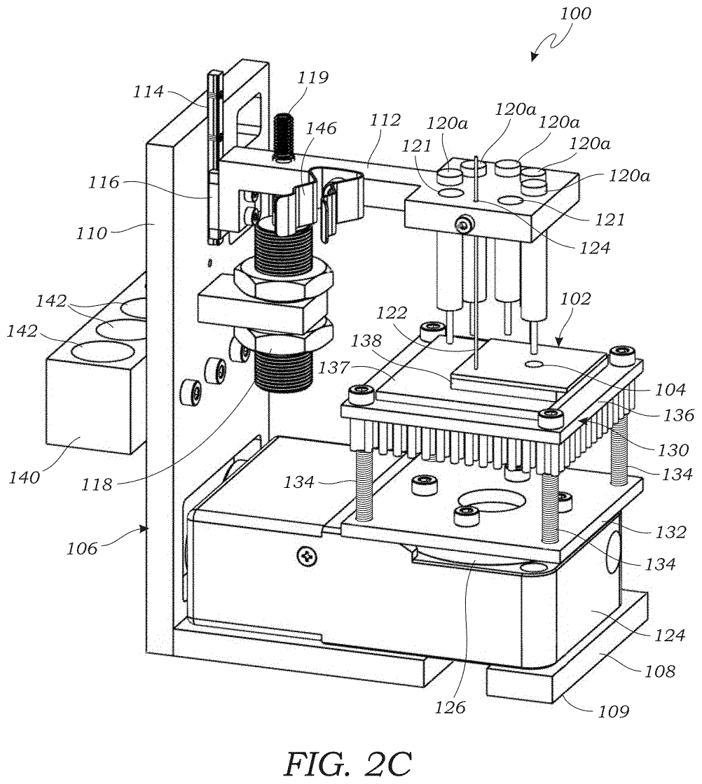

Referring to FIGS. 2A, 2B and 2C, embodiments of a radiosynthesis device 100 according to the present disclosure are illustrated. The radiosynthesis device 100 is configured to perform a micro-droplet based chemical synthesis, such as a radiosynthesis to produce PET tracers used in positron emission tomography. Although the radiosynthesis device 100 is described herein for producing radiochemicals, the devices and methods disclosed herein may be used for any suitable chemical synthesis. For example, the system can be used to make small batches of novel compounds (e.g., for evaluation in some kind of assay), or for preparing small amounts of short-lived materials that cannot be produced in large batches, or are difficult or expensive to produce in large batches. Furthermore, the system can be used in complex multi-step syntheses of novel compounds, e.g., when it is unclear how to set the reaction parameters for one step of the synthesis—in this case the novel system allows attempts of the reaction at one or more conditions, while consuming very little of the total material, to help guide how to optimally perform the next step of the reaction.

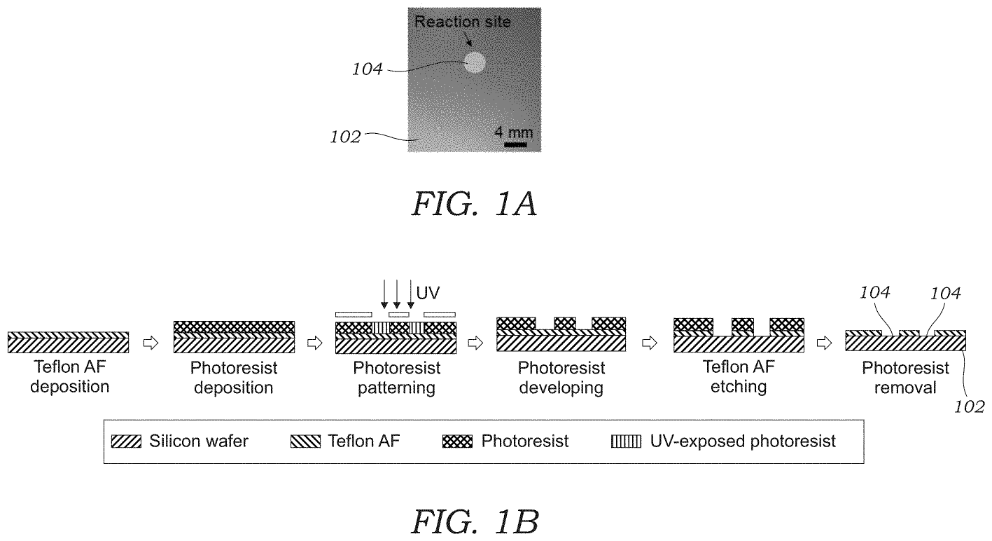

The radiosynthesis device 100 is configured to utilize a microfluidic chip 102 having one or more reaction sites 104, as also shown in FIGS. 1A, 1B, 9A and 9B. As shown in FIGS. 2A, 2B and 2C, the radiosynthesis device 100 includes a support frame or housing 106 that holds the various components of the radiosynthesis device 100. The support frame 106 has a base 108. The base 108 may be a rectangular shaped plate, or other suitable shape. The base 108 is oriented horizontally and has a flat bottom surface 109 such that it can stably sit on a supporting surface such a lab benchtop or table. The frame 106 also has a support wall 110 connected to, and extending upward from, one side of the base 108. A support arm 112 (also referred to herein as a “fixture”) is slidably coupled to the support wall 110 and extends horizontally from the support wall 110 over the base 108. The support arm 112 is moveable up and down in the vertical direction. The support arm 112 is slidably coupled to the support wall 110 using a vertically oriented rail 114 attached to the support wall 110 which slidably receives a raceway of a slide 116 attached to the support arm 112. The support arm 112 has a plurality of dispenser receiving apertures 121 for receiving and holding dispensers 120. The dispenser receiving apertures 121 are arranged in angularly spaced apart relation along an arc of a circle having a center point. In the illustrated embodiment of FIGS. 2B and 2C, dispenser receiving apertures 121 are angularly spaced apart 45°, and two of dispenser receiving apertures 121 do not have dispensers 120 installed in them. Depending on the particular radiosynthesis process being performed on the radiosynthesis device 100, more or fewer dispensers 120 may be required.

A pneumatic cylinder 118 (e.g., a single-acting pneumatic cylinder) is attached to the support wall 110 and has an actuator rod 119 (e.g., a piston rod of a single-acting pneumatic cylinder) connected to the support arm 112. The actuator 118 is controllably actuatable to move the support arm 112 up and down relative to the microfluidic chip 102 in order enable easy loading and unloading of the microfluidic chip, and/or to adjust the vertical position of the dispensers 120 and collection tube 122 relative to the reaction site 104 on the microfluidic chip 102.

A plurality of non-contact dispensers 120 are installed on the support arm 112 of the frame 106 (inserted into and/or affixed to the dispenser receiving apertures 121), including a first dispenser 120a, a second dispenser 120b, a third dispenser 120c, a fourth dispenser 120d and a fifth dispenser 120e. The dispensers 120 extend downward from the support arm 112 above the microfluidic chip 102. The non-contact dispensers 120 are typically solenoid-based, non-contact fluid dispensers, but may be any suitable dispenser for dispensing the reagents utilized in a desired radiosynthesis process. The dispensers 120 may have metal components (nozzles), but such metal components may be susceptible to attack by acidic reagents. Hence, the metal nozzles may be cleaned and/or coated and/or made out of other materials (e.g., plastic) to improve the lifetime. In addition, disposable dispensers may be utilized, or dispensers having nozzles which are not degraded by the reagents, such as acidic reagents.

The dispensers 120 are arranged in angularly spaced apart relation along an arc of a circle having a center point. In the illustrated embodiment of FIGS. 2B and 2C, the dispensers 120 are angularly spaced apart 45°.

A collection tube 122 is also installed on the support arm 112 of the frame 106. The collection tube 122 inserts into and is affixed through a tube aperture 124 in the support arm 112. The collection tube 122 extends downward from the support arm 112 above the microfluidic chip 102, and terminates just above (e.g., about 0.5 mm or less) the surface of microfluidic chip 102. The collection tube 122 is also positioned in angularly spaced apart relation from the dispensers 120 along the same arc of a circle as the dispensers 120. In the illustrated embodiment of FIGS. 2B and 2C, the collection tube 122 is angularly spaced apart from the dispenser 102e by 90° and by 45° from the directly adjacent dispenser receiving apertures 121 (there is one empty dispenser receiving apertures 121 between the collection tube 122 and the directly adjacent dispenser receiving apertures 121).

The radiosynthesis device 100 may be configured to perform multiple different syntheses on the same microfluidic chip 102 having multiple reaction sites 104, with each different syntheses in a separate reaction site 104. For example, different tracers or probes could be produced on the same microfluidic chip 102 on the same radiosynthesis device 100. This may require adding more dispensers 120, which could be accommodated by increasing the radius of the arc upon which the dispensers 120 are positioned, and also increasing the radius of the arc upon which the reaction sites 104 are positioned on the microfluidic chip 102. In addition, multiple collection tubes 122 can be added to the support arm 112, each of which is connected to a separate purification/formulation system and/or collection container 148.

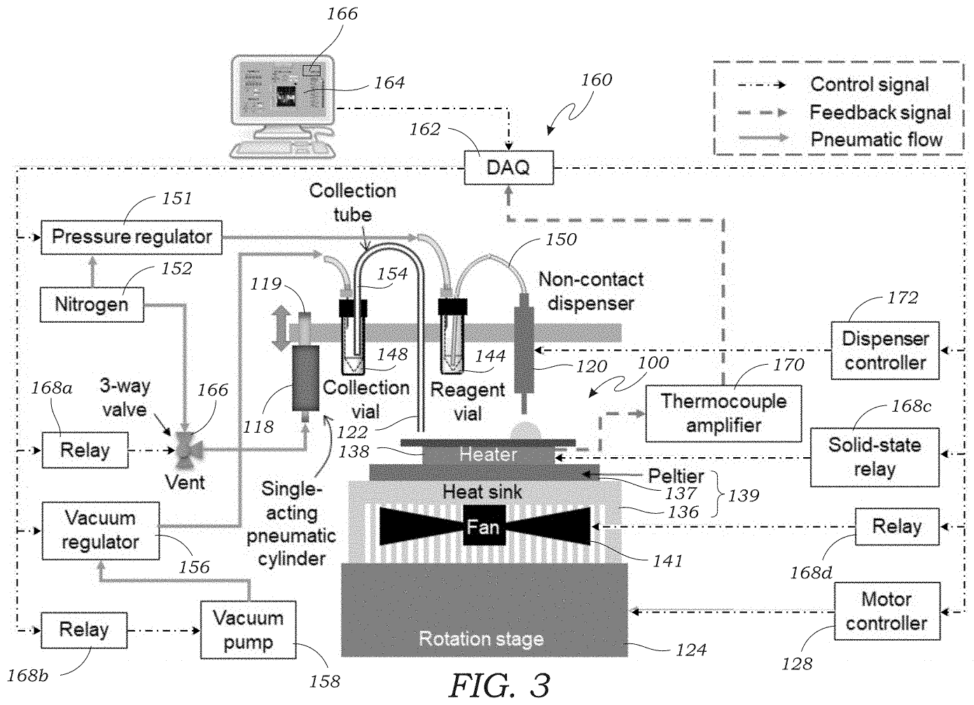

The radiosynthesis device 102 also has a motorized rotation stage 124 mounted on the top of the base 108. The motorized rotation stage 124 has a controllably rotatable platform 126. The motorized rotation stage 124 accurately rotates the rotatable platform 126 based on a control signal from a motor controller 128 (see FIG. 3).

A thermally controlled support 130 is coupled to the rotatable platform 126 of the motorized rotation stage 124, such that rotation of the rotatable platform 126 rotates the thermally controlled support 130. The thermally controlled support 130 includes a support base 132 which is mounted to the rotatable platform 126, a plurality of risers 134 which are attached to the base 132 and extending upward from the support base 132. The illustrated embodiments of FIGS. 2A, 2B and 2C have four risers 134, but any suitable number of risers 134 may be used. A support platform/heat sink 136 is mounted on top of the risers 134. A thermoelectric cooler 137 (e.g., a Peltier cooling device) is mounted on the top of the heatsink 136, and a fan 141 (see FIGS. 2B and 3) is mounted on the bottom surface of the heat sink 136. The thermoelectric cooler 137 is in thermal contact with the heat sink 136 and the microfluidic chip 102. The thermoelectric cooler 137, heatsink 136 and fan 141 may be integrated as an integrated cooling module 139. A heater element 138 (e.g., a ceramic heater) is mounted on top of the thermoelectric cooler 137, and the microfluidic chip 102 sits on the heater element 138, or on a chip holder mounted on the heater 138. The heater element 138 is also in thermal contact with the microfluidic chip 102.

The heater element 138 may include positioning element(s), such as a recess, bumps, guides, etc., or a chip holder having such positioning element(s), for accurately positioning and/or securing the microfluidic chip 102 on the thermally controlled support 130. The thermally controlled support 130 may hold the microfluidic chip 102 such that the reaction site(s) 104 are off-center with respect to the axis of rotation of the motorized rotation stage 124 (and support 130, which has the same axis of rotation) so that the reaction site(s) 104 move through an arc when the support 130 is rotated (as opposed to a reaction site 104 position with its center on the axis of rotation in which case the reaction site 104 merely rotates about its center).

A reagent container rack 140 is mounted on the outside surface of the support wall 110. The reagent container rack 140 has a plurality of holes 142 for receiving and holding reagent containers 144 (e.g., reagent vials 144) (see FIG. 3).

A collection container holder 146 (e.g., a vial clip) is attached to the support arm 112 of the fixture for holding a collection container 148 (e.g., a collection vial 148) (see FIG. 3). A collection tube 154 fluidly connects the collection tube 122 to the collection vial 148. The collection tube vial 148 may be placed anywhere, or it can be located inside a “pig” so that once the reaction product is delivered into the vial 148, it can be safely handled by an operator.

Turning to FIGS. 2B and 3, a control system 160 and fluid connections for controlling the operations of the radiosynthesis device 100 will now be described. Each of the reagent containers 144 are in fluid communication with a respective dispenser 120 via reagent tubes 150 (150a, 150b and 150c). In addition, each of the reagent containers 144 is pressurized via a pressure regulator 151 and a pressure source 152 (e.g., pressurized nitrogen) for delivering droplets of reagent from the reagent containers 144 upon actuation of the dispensers 120. The pressure regulator 151 is operably coupled to a control system 160 to electronically control the pressure supplied to the dispensers 120. As shown in FIG. 3, the pressure regulator 151 is connected to the control system 160 via a data acquisition device 162 which provides an interface between the pressure regulator 151 and a computing device 164 (e.g., a personal computer or other suitable computer) executing a lab systems control software program 166 (e.g., LabView from National Instruments). Each of the non-contact dispensers 120 is operably coupled to a dispenser controller 172 which is in turn connected to the computing device 164 of the control system 160 via the data acquisition device 162, to independently control the operation of each of the dispensers 120 to dispense droplets of reagent from each dispenser 120.

The pneumatic cylinder 118 is operably connected to a 3-way valve 166 which is connected to the pressure source 152 to provide actuation pressure to the pneumatic cylinder 118. The 3-way valve 166 is operably coupled to a first relay 168a which is in turn operably connected to the computing device 164 of the control system 160 via the data acquisition device 162, to control the operation of the 3-way valve 166. The 3-way valve 166 is actuatable by the control system 162 to pressurize the pneumatic cylinder 118 from the pressure source 152 or to vent the pressure in the pneumatic cylinder 118, in order to actuate and de-actuate the pneumatic cylinder 118 to move the actuator rod 119 up and down, which in turn moves the support arm 112, dispensers 120 and collection tube 122 up and down.

The heater element 138 is operably coupled to a solid-state relay 168c which is in turn operably connected to the computing device 164 of the control system 160 via the data acquisition device 162, to control the operation of the heater element 138. The heater element 138 is also coupled to a thermocouple amplifier 170 which is in turn operably connected to the computing device 164 of the control system 160 via the data acquisition device 162, to provide temperature feedback control of the heater element 138. Similarly, the thermoelectric cooler 137 and fan 141 are operably coupled to a relay 168d which is in turn operably connected to the computing device 164 of the control system 160 via the data acquisition device 162, to control the operation of the thermoelectric cooler 137 and the fan 141.

The collection container 148 is in fluid communication with the collection tube 122 via a collection container tube 154. The collection container 148 is also in fluid communication with a vacuum regulator 156 and a vacuum source 158 (e.g., a vacuum pump) for withdrawing droplets of reaction product from the reaction site 104 into the collection tube 122 and into the collection container 148. The vacuum source 158 is operably coupled to a second relay 168b which is in turn operably connected to the computing device 164 of the control system 160 via the data acquisition device 162, to control the operation of the vacuum source 158.

The motorized rotation stage 124 is operably coupled to the motor controller 128 which is in turn connected to the computing device 164 of the control system via the data acquisition device 162, to control the operation (i.e., rotation) of the motorized rotation stage 124.

As mentioned above, the control system 160 includes a computing device 164 having a lab systems control software program 166, such as LabView. The data acquisition device 162 provides an interface between the computing device 164 and each of the control elements (e.g., the relays 168, 3-way valve 166, dispenser controller 172, motor controller 128, etc.) and receives and processes the feedback signals (e.g., signal from thermocouple amplifier 170, etc.). Accordingly, the computing device 164 executing the lab systems control software program 166 can automatically control: the rotation of the motorized rotation stage 124; the dispensing of reagents from the dispensers 120; the operation of the heater element 138 and thermoelectric cooler 137 to control the temperature of the microfluidic chip 102; the operation of the vacuum source 158 to withdraw reaction product or intermediates synthesized on the microfluidic chip 102; the operation of the pneumatic cylinder 118 to raise and lower the support arm 112; and any other functions of the radiosynthesis device 100. The lab systems control software program 166 is programmable to control the operation of the radiosynthesis device 100 automatically via a program or series of operations that are stored or accessed by the software 166 such that no human involvement is needed except for the loading and unloading of the microfluidic chip 102. In other embodiments, one or more operations may require some manual input or intervention.

Referring to FIG. 2A, the perspective solid view of the radiosynthesis device 100 is shown alongside a 12 oz. coffee cup 103 to illustrate the small size of the radiosynthesis device 100. The radiosynthesis devices 100 as disclosed herein have dimensions of no more than 10 cm×6 cm×12 cm (width×depth×height), or about 750 cm³.

Turning to FIGS. 1A and 1B, an exemplary microfluidic chip 102 and process for fabricating the microfluidic chip 102 are illustrated. The microfluidic chip 102 has a single circular, hydrophobic reaction site having a diameter of 104. FIG. 1B illustrates one exemplary photolithography process for the fabricating the microfluidic chip 102. Other suitable fabrication processes may be utilized to manufacture the microfluidic chip 102. It should be appreciated that in other embodiments, multiple reaction sites 104 can be formed on a single microfluidic chip, as shown in FIG. 9A. A shown in FIG. 9A, the multiple reaction sites 104 are positioned about an arc of a circle on the microfluidic chip 102.

As shown in FIG. 1B, the photolithography process for fabricating the microfluidic chip 102 includes: Teflon AF deposition on a silicon wafer; depositing photoresist onto the Teflon AF coated silicon wafer; patterning the photoresist in the form of the desired reaction site(s) 104; developing the photoresist; etching away the Teflon AF to form the silicon (i.e., hydrophilic) reaction site(s) 104; and removing the photoresist. The resulting product is a microfluidic chip 102 having one or more reaction sites 104.

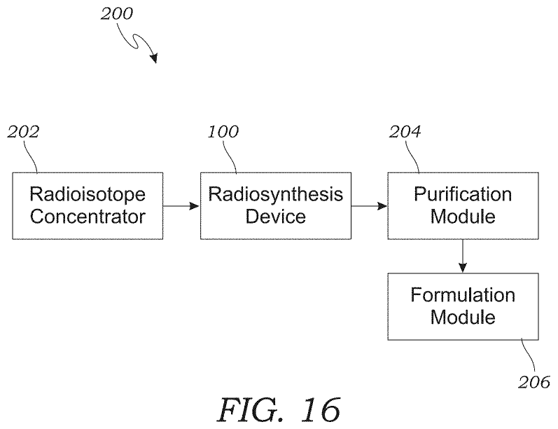

Turning to FIG. 16, a complete radiosynthesis system 200 utilizing the radiosynthesis device 100 is illustrated of FIG. 2A. The radiosynthesis system 200 includes a radionuclide concentrator 202 (also referred to as a radioisotope concentrator) connected to the radiosynthesis device 100 upstream of the radiosynthesis device 100. The radionuclide concentrator 202 is configured to concentrate a radioisotope and output the radioisotope to the radiosynthesis device 100. For instance, the radionuclide concentrator 202 may be a micro-cartridge based radionuclide concentrator. This increases the amount of radioactivity used in the synthesis process, and can produce [¹⁸F]fallypride, or other PET traces, at the GBq level. The radiosynthesis system 200 also has a purification module 204 and a formulation module 206 connected to the radiosynthesis device 100 downstream of the radiosynthesis device 100. As some examples, the purification module 204 may be an analytical-scale HPLC system or a cartridge purification system. By integrating the radionuclide concentrator with the radiosynthesis device 100, it is much easier and faster to scale up the synthesis to clinically-relevant levels.

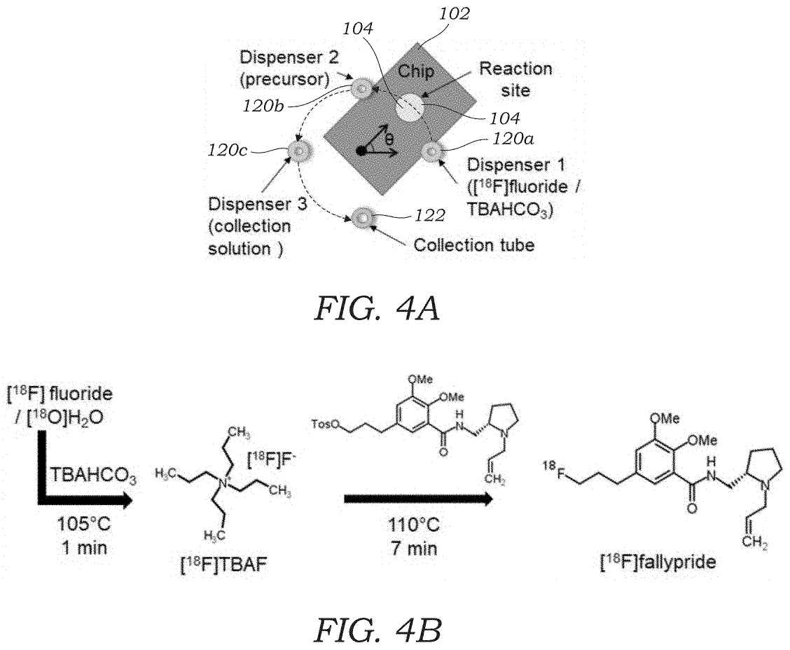

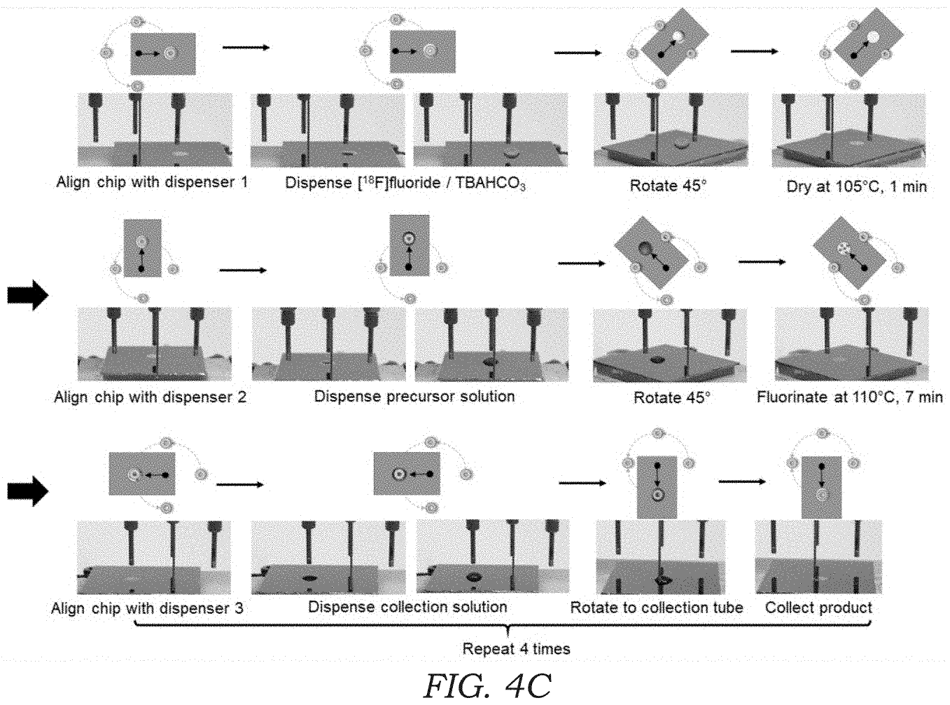

Referring to FIGS. 4A-4C, an exemplary method of using the radiosynthesis device 100 to perform a synthesis process to synthesize a chemical product will now be described. The method of using the radiosynthesis device 100 shown in FIGS. 4A-4C is for synthesizing [¹⁸F]fallypride, but the method is not limited to only synthesizing [¹⁸F]fallypride. Instead the method can be used to synthesize any suitable chemical, in some cases, with modifications within the ordinary skill in the art.

As shown in FIG. 4A, the radiosynthesis device 100 is configured with three dispensers 120, a first dispenser 120a (radioisotope dispenser), a second dispenser 120b (precursor dispenser), and a third dispenser 120c (collection solution dispenser), and the collection tube 122, angularly spaced apart 90° along an arc of a circle. All of the operations are controlled by the computing device 164 executing the lab systems control software program 166. First, the motorized rotation stage 124 rotates the microfluidic chip 102 to position the reaction site 104 at the first dispenser 120a. The first dispenser 120a dispenses one or more droplets of a radioisotope stock solution comprising a radioisotope in a solvent onto the reaction site 104. Next, the motorized rotation stage 124 rotates the microfluidic chip 102 by 45° CCW. At this position, the radioisotope stock solution on the first reaction site 102 is heated using the heater element 138 of the thermally controlled support 130 to evaporate the solvent leaving a dried residue of radioisotope complex on the reaction site 104. Then, the microfluidic chip 102 is rotated 45° CCW by rotating the motorized rotation stage 124 to position the reaction site 104 at the second dispenser 120b. The second dispenser 120b dispenses one or more droplets of precursor solution onto the reaction site 104 to dissolve the dried residue of radioisotope complex resulting in a solution of precursor solution and radioisotope complex. The microfluidic chip 102 is rotated 45° CCW by rotating the motorized rotation stage 124, and the chip 102 is heated using the heater element 138 of the thermally controlled support 130 to perform a radiofluorination reaction resulting in crude radiochemical product. Next, the microfluidic chip is rotated 45° CCW by rotating the motorized rotation stage 124 to position the reaction site 102 at the third dispenser 120c. The third dispenser 120c dispenses one or more droplets of collection solution onto the reaction site 102 containing crude radiochemical product to dilute the crude radiochemical product. Then, the microfluidic chip 102 is rotated 90° CCW by rotating the motorized rotation stage to position the reaction site 102 at the collection tube 122. Then, the diluted crude radiochemical product is removed from the reaction site 102 using the collection tube by applying a vacuum from the vacuum source 158 to the collection container 148 and the collection tube 122. The microfluidic chip 102 is then rotated 90° CW back to the third dispenser 120c, the third dispenser 120c dispenses more collection solution onto the reaction site 104, the microfluidic chip 102 is rotated 90° CCW to the collection tube 122, and additional diluted crude product is withdrawn into the collection tube 122 and into the collection container 148. This collection process is repeated four times, or any other suitable number of times, such as two times, three times, five times, or more.

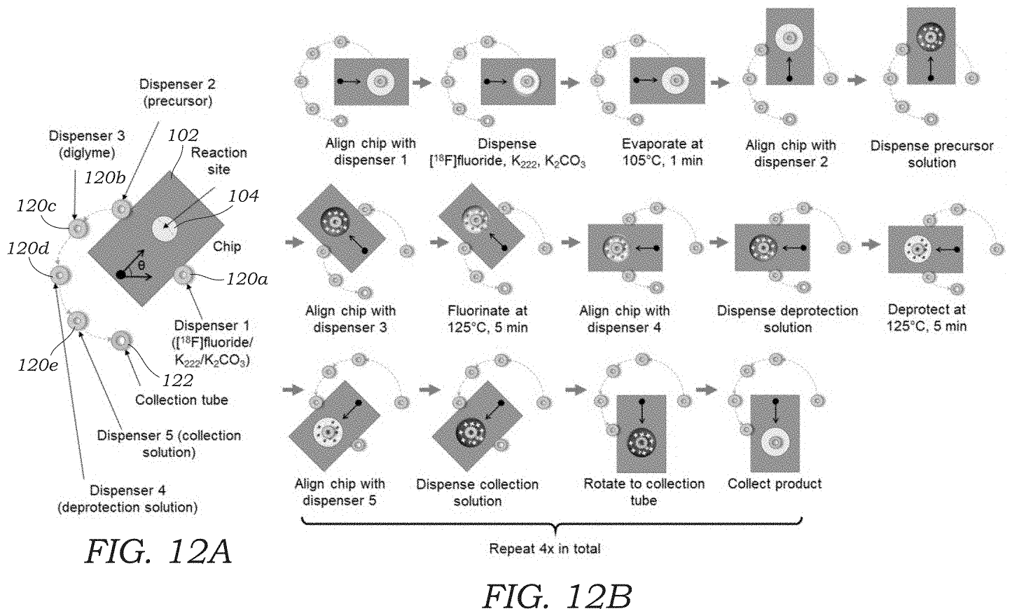

Referring to FIGS. 12A-12B, another method of using the radiosynthesis device 100 to perform a synthesis process to synthesize a chemical product is illustrated. The method of FIGS. 12A-12B is similar to the method shown in FIGS. 4A-4C, except that the radiosynthesis device 102 used in the method of FIGS. 12A-12B includes five dispensers 120 which dispense five different reagents, and the method includes several additional steps. In addition, the first dispenser 120a is angularly spaced apart from the second dispenser 120b by 90°; and the second dispenser 120b, third dispenser 120c, fourth dispenser 120d, fifth dispenser 120e and collection tube 122 are angularly spaced apart by 45°. The synthesis method shown in FIGS. 12A-12B is for synthesizing [¹⁸F]FDOPA, but the basic method is not limited to only synthesizing [¹⁸F]FDOPA. Instead the method can be used to synthesize any suitable chemical, in some cases, with modifications within the ordinary skill in the art. In view of the description of the operation of the radiosynthesis device 102 to perform the method shown in FIGS. 4A-4C, and the description of the specific synthesis of [¹⁸F]FDOPA in the Examples below, the operation of the radiosynthesis device 102 to perform the method shown in FIGS. 12A-12B, is self-explanatory.

### EXPERIMENTAL EXAMPLES

The following examples, and corresponding figures demonstrate the use of the radiosynthesis device 100, and microfluidic chip 102 to synthesize various PET tracers.

Materials and Methods

Materials

Anhydrous acetonitrile (MeCN, 99.8%), methanol (MeOH), 2,3-dimethyl-2-butanol (thexyl alcohol, 98%), trimethylamine (TEA), ammonium formate (NH₄HCO₂; 97%) were purchased from Sigma-Aldrich. Tetrabutylammounium bicarbonate (TBAHCO₃, 75 mM), tosyl fallypride (fallypride precursor, >90%) and fallypride (reference standard for [¹⁸F]fallypride, >95%) were purchased from ABX Advanced Biochemical Compounds (Radeberg, Germany). Food dye was purchased from Kroger (Cincinnati, Ohio, USA) and diluted with solvents in the ratio of 1:100 (v/v) to perform a mock synthesis. DI water was obtained from a Milli-Q water purification system (EMD Millipore Corporation, Berlin, Germany). No-carrier-added [¹⁸F]fluoride in [¹⁸O]H₂O was obtained from the UCLA Ahmanson Biomedical Cyclotron Facility.

Apparatus

Reactions were performed on microfluidic chips 102 (also referred to as “chip 102”), as illustrated in FIGS. 1-4, each comprising a hydrophilic circular reaction site 104 (4 mm diameter) patterned in the hydrophobic Teflon® AF surface of a silicon chip (25 mm×27.5 mm). The patterned chips were prepared by coating silicon wafers with Teflon® AF, and then etching away the coating to leave the desired hydrophilic pattern as described previously (see, e.g., J. Wang, P. H. Chao, S. Hanet and R. M. van Dam, Lab. Chip, 2017, 17, 4342-4355). For this work, we omitted the final Piranha cleaning step. Chips were used once each and then discarded after use.

Operations on the microfluidic chip 102 were automated by a custom-built compact radiosynthesis device 100 as shown in FIGS. 2A, 2B, 2C and 3 comprising a rotating, temperature-controlled platform 130, a set of reagent dispensers 120, and a collection system 122, 148 to remove the reaction droplet at the end of the synthesis. The control system is as shown in FIG. 3.

Heating was provided by placing the microfluidic chip 102 in direct contact with a 25 mm×25 mm ceramic heater (Ultramic CER-1-01-00093, Watlow, St. Louis, Mo., USA). A thin layer of thermal conducting paste (OT-201-2, OMEGA, Norwalk, Conn., USA) was applied between the chip and heater to improve heat transfer. The chips could easily be aligned during installation by lining up three edges of the chip with the edges of the heater. The heater was glued atop a 40 mm×40 mm thermoelectric device (Peltier, VT-199-1.4-0.8, TE Technology, Traverse City, Mich., USA) mounted to a 52 mm×52 mm integrated heatsink and fan (4-202004UA76153, Cool Innovations, Concord, Canada) (“integrated cooling module 139”). The integrated cooling module 139 was mounted via a custom aluminum plate to a motorized rotation stage (OSMS-40YAW, OptoSigma, Santa Ana, Calif., USA). The signal from a K-type thermocouple embedded in the heater was amplified through a K-type thermocouple amplifier (AD595CQ, Analog Devices, Norwood, Mass., USA) and connected to an analog input of the data acquisition device (DAQ; NI USB-6003, National Instruments, Austin, Tex., USA). The power supply (120 V AC) for the heater was controlled by a solid-state relay (SSR, Model 120D25, Opto 22, Temecula, Calif., USA) driven by a digital output of the DAQ. An on-off temperature controller was programmed in LabView (National Instruments) to maintain a desired setpoint. A power step down module (2596 SDC, Model 180057, DROK, Guangzhou, China) was connected to a 24V power supply to provide 12V for the cooling fan, which was switched on during cooling via an electromechanical relay (EMR, SRD-05 VDC-SL-C, Songle Relay, Yuyao city, Zhejiang, China) controlled by the LabView program. The motorized stage was driven by a stage controller (GSC-01, OptoSigma) controlled by the LabView through serial communication.

Droplets were loaded at the reaction site 104 of the microfluidic chip 102 through miniature, solenoid-based, non-contact dispensers 120. Chemically-inert dispensers with FFKM seal (INKX0514100A, Lee Company, Westbrook, Conn., USA) were used for reagents containing organic solvents, while a dispenser with EPDM seal (INKX0514300A, Lee Company) was utilized to dispense [¹⁸F]fluoride solution. Each dispenser 120 was connected to a pressurized vial of a reagent and the internal solenoid valve was opened momentarily to dispense liquid. More details of the fluidic connections are described above. Each dispenser 120 was connected to a dedicated controller (IECX0501350A, Lee Company), driven by a digital output from the DAQ 162 and controlled via the LabView program 166. Since the volume of dispensed liquid is related to the driving pressure, the opening duration of the valve, and physical properties (e.g., viscosity) of the solvent, calibration curves were generated for each reagent as described previously (see, e.g., J. Wang, P. H. Chao, S. Hanet and R. M. van Dam, Lab. Chip, 2017, 17, 4342-4355).

A fixture 112 was built to hold up to 7 dispensers 120 with nozzles located ˜3 mm above the chip 102. Each dispenser 120 was secured within a hole by an O-ring (ORBN005, Buna-N size 005, Sur-Seal Corporation, Cincinnati, Ohio, USA). The fixture 112 was mounted to a vertically-oriented movable slide 116, and a single-acting air cylinder 118 (6604K13, McMaster-Carr) was configured to allow the fixture 112 to be raised 16 mm above the surface to facilitate installation and removal of microfluidic chips 102 and cleaning of the dispensers 120. The air cylinder 118 was connected to a 3-way valve 166 (LVM105R-2, SMC Corporation) to apply either pressure (˜210 kPa [˜30 psi]) or vent to atmosphere, the valve 166 was controlled by a LabView software program.

The heater 138 and chip 102 were mounted off-center of the rotation axis of motorized rotation stage 124 and thermally controlled support 130. During multi-step reactions, the chip 102 was rotated to position the reaction site 104 underneath a dispenser 120 to add the desired reagent, and was then rotated to a position in between dispensers 120 while performing evaporations or reactions at elevated temperatures.

To transfer the final crude product from the reaction site 104 on the chip 102 to the collection vial 148, a metal tubing (0.25 mm inner diameter) was mounted in the dispenser fixture 112 such that the end was ˜0.5 mm above the chip surface. At the end of synthesis, the platform 130 was rotated such that the reaction droplet was aligned under the collection tube 122 and vacuum was applied to the headspace of the collection vial using a compact vacuum pump 158 (0-16″ Hg vacuum range, D2028, Airpon, Ningbo, China) connected via a vacuum regulator 156 (ITV0090-3UBL, SMC Corporation) controlled via the LabView program. Vacuum pressure was ramped from 0 to 14 kPa (˜2 psi, 0.01 psi increment every 50 ms) over 10 s to transfer the droplet into the collection vial 148.

After the synthesis, dispensers 120 were each cleaned by flushing with DI water (1 mL) and MeOH (1 mL) in sequence, driven at 69 kPa [˜10 psi], and then drying with nitrogen for 2 min. The used chip 102 was removed with tweezers and discarded.

Automated Droplet Synthesis of [¹⁸F]Fallypride

As a model reaction to demonstrate the ability to perform multi-step reactions automatically with the microdroplet radiosynthesizer, syntheses of the PET tracer [¹⁸F]fallypride was performed. The synthesis protocol was adapted from a manual synthesis protocol developed via manual optimization efforts using microfluidic chips having a similar circular hydrophilic reaction zone (see, e.g., Rios, A., Wang, J., Chao, P. H., & van Dam, R. M. (2019). A novel multi-reaction microdroplet platform for rapid radiochemistry optimization. RSC Advances, 9(35), 20370-20374).

A [¹⁸F]fluoride stock solution was prepared by mixing [¹⁸F]fluoride/[¹⁸O]H₂O (60 μL, ˜110 MBq [˜3 mCi]) with 75 mM TBAHCO₃ solution (40 μL). The final TBAHCO₃ concentration was 30 mM. Precursor stock solution was prepared by dissolving tosyl-fallypride precursor (2 mg) in a mixture of MeCN and thexyl alcohol (1:1 v/v, 100 μL) to result in a final concentration of 39 mM. A stock solution for dilution of the crude product prior to collection was prepared from a mixture of MeOH and DI water (9:1, v/v, 500 μL). These solutions were loaded into individual reagent vials connected to dispensers.

To carry out the synthesis on the chip, the chip was first rotated to position the reaction site below the [¹⁸F]fluoride/TBAHCO₃ dispenser and eight 1 μL droplets of [¹⁸F]fluoride/TBAHCO₃ solution (˜8.9 MBq; ˜0.24 mCi) were sequentially loaded onto the chip (total time <10 s). The chip was rotated 45° counterclockwise (CCW) and heated to 105° C. for 1 min to evaporate the solvent and leave a dried residue of the [¹⁸F]TBAF complex at the reaction site. Then, the chip was rotated 45° CCW to position the reaction site under the precursor dispenser and twelve 0.5 μL droplets of precursor solution were loaded to dissolve the dried residue. Next, the chip was rotated 45° CCW and heated to 110° C. for 7 min to perform the radiofluorination reaction. Afterwards, the chip was rotated 45° CCW to position the reaction site under the collection solution dispenser, and twenty 1 μL droplets of collection solution were deposited to dilute the crude product. After rotating the chip 90° CCW to position the reaction site under the collection tube, the diluted solution was transferred into the collection vial by applying vacuum. The collection process was repeated a total of four times to minimize the residue on the chip (i.e., by rotating the chip 90° CW back to the collection solution dispenser, loading more collection solution, etc.). A schematic of the whole synthesis process is shown in FIGS. 4A-4C.

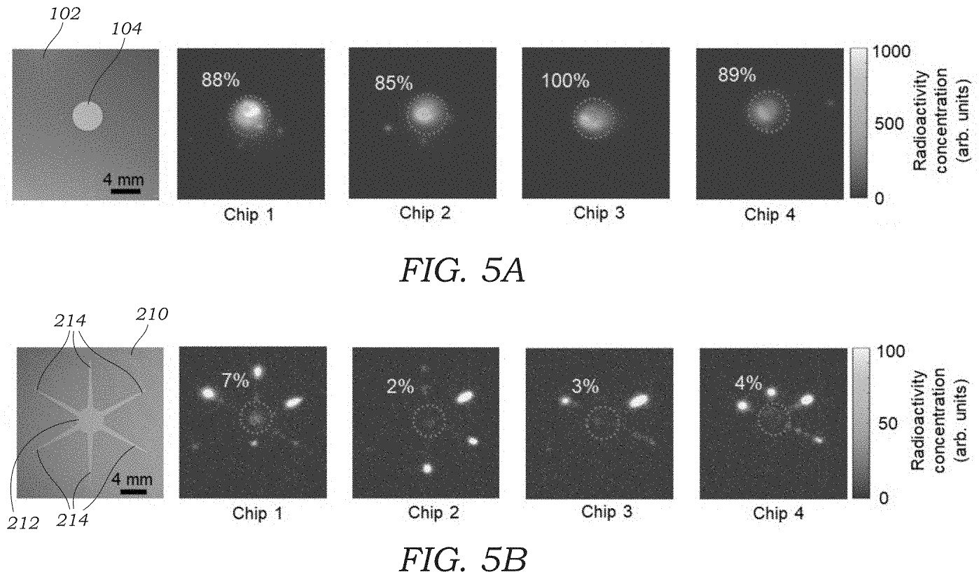

To compare the performance of the new setup to previous work, the same [¹⁸F]fallypride synthesis conditions were implemented on the previous “passive transport” chip. The chip 210 was composed of one hydrophilic 4 mm reaction site 212 and six radial, tapered, hydrophilic fluid delivery channels 214 (FIG. 5B), and reagent delivery and production collection were performed as previously described.

Analytical Methods

Performance of the [¹⁸F]fallypride synthesis on the chip was assessed through measurements of radioactivity and fluorination efficiency. Radioactivity was measured with a calibrated dose calibrator (CRC-25R) at various times throughout the synthesis process, including starting radioactivity on the chip after loading of [¹⁸F]fluoride/TBAHCO₃ stock solution, radioactivity of crude product transferred into the collection vial and radioactivity of residue on the chip after collection step. Radioactivity recovery was calculated as the ratio of radioactivity of collected crude product to starting radioactivity on the chip. Residual activity on the chip was the ratio of radioactivity on the chip after collection to the starting radioactivity on the chip. All measurements were corrected for decay.

Fluorination efficiency of the crude product collected from the chip was determined via radio thin layer chromatography (radio-TLC). A 1 μL droplet was spotted on a silica gel 60 F₂₅₄ sheets (aluminum backing) with a micropipette. The TLC plate was dried in air and developed in the mobile phase of 60% MeCN in 25 mM NH₄HCO₂ with 1% TEA (v/v), and then analyzed with a scanner (MiniGITA star, Raytest, Straubenhardt, Germany). The resulting chromatograms showed peaks corresponding to unreacted [¹⁸F]fluoride (Rf=0.0) and [¹⁸F]fallypride (Rf=0.9). Fluorination efficiency was calculated as the peak area of the [¹⁸F]fallypride peak divided by the area of both peaks. Crude radiochemical yield (crude RCY, decay-corrected) was defined as the radioactivity recovery times the fluorination efficiency.

In some cases, radio-HPLC purification of the collected crude product was carried out using a Smartline HPLC system (Knauer, Berlin, Germany) equipped with a degasser (Model 5050), pump (Model 1000), a UV (254 nm) detector (Eckert & Ziegler, Berlin, Germany) and a gamma-radiation detector and counter (B-FC-4100 and BFC-1000; Bioscan, Inc., Poway, Calif., USA). Separation was performed using an analytical C18 column (Kinetex, 250×4.6 mm, 5 μm, Phenomenex) with mobile phase (60% MeCN in 25 mM NH₄HCO₂ with 1% TEA (v/v)) at 1.5 mL/min flow rate. The crude product collected from the chip was injected into the HPLC system, and the [¹⁸F]fallypride fraction (˜2 mL) was collected (retention time ˜4.5 min). Chromatograms were recorded using a GinaStar analog-to-digital converter (raytest USA, Inc., Wilmington, N.C., USA) and GinaStar software (raytest USA, Inc.) running on a PC. The collected product fraction was then dried by evaporation of solvent in an oil bath at 110° C. for 8 min with nitrogen flow, and then redissolved in PBS. The purity and identity of the purified [¹⁸F]fallypride was verified using the same HPLC system and conditions.

For the experiments that included the purification step, the radioactivity of purified product recovered from HPLC was also measured. The purification efficiency was calculated by dividing the radioactivity of the purified product by the radioactivity of the collected crude product. RCY was defined as the ratio of radioactivity of the purified product to the starting radioactivity on the chip.

To visualize the distribution of radioactivity on the chips, a custom Cerenkov Luminescence Imaging (CLI) setup was used. In particular, the visualization focused on imaging after the collection step. To acquire an image, the chip was placed in a light-tight box, covered with a plastic scintillator (1 mm thick) to increase the luminescence signal, and imaged for 300 s. After acquisition, the raw image was processed via image correction and background correction steps as described previously. To analyze the ratio of residual activity within the area of the reaction site to the total residual activity on the chip (i.e., reaction site and surrounding region), regions of interests (ROIs) were drawn to encircle both the reaction site and the whole chip. The desired ratio was calculated as the sum of pixel values within the reaction site ROI divided by sum of pixel values within the whole chip ROI.

Results and Discussion

Mock Radiosyntheses

To test the feasibility of multi-step reactions on the microdroplet radiosynthesizer, a mock synthesis of [¹⁸F]fallypride was performed first, in which [¹⁸F]fluoride/TBAHCO₃ solution was replaced with DI water, and precursor solution was replaced with the solvent mixture only. Diluted food dyes of different colors were added in each solution: yellow dye was mixed with DI water, red dye was mixed with a mixture of MeCN and thexyl alcohol (1:1, v/v), and blue dye was mixed with a mixture of MeOH and DI water (9:1, v/v). To dispense these solutions, reagent reservoirs were pressurized to ˜35 kPa [˜5 psi] and an opening duration of 1.0 ms was used. The synthesis scheme and a series of photographs of the overall process is shown in FIG. 4. During the mock synthesis, it was observed that the rotation stage moved the chip quickly and accurately to each desired position, the reagents were accurately delivered to the reaction sites without any visible splashing, and the solutions on the chip remained confined to the reaction site during all steps of the synthesis process.

[¹⁸F]Fallypride Synthesis

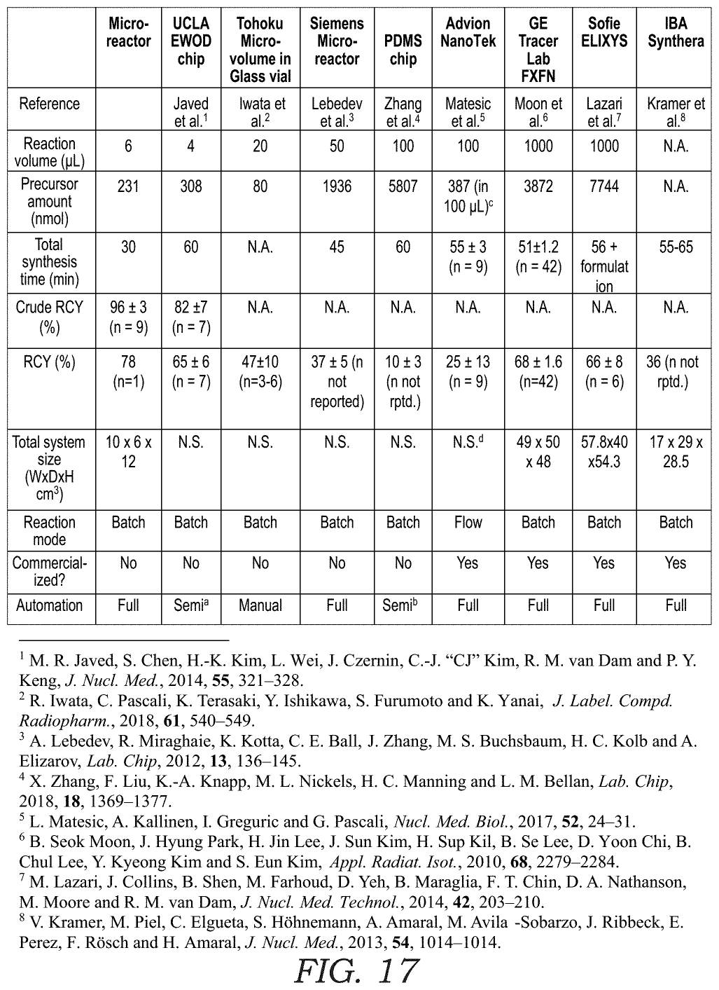

To evaluate the performance and consistency of the [¹⁸F]fallypride syntheses, multiple radiosynthesis per day were performed on two separate days (see FIG. 17, Table 1). Overall, the crude RCY was very high and was consistent across the two days (95±3% (n=5) for day 1 and 97±2% (n=4) for day 2). The fluorination efficiency was very consistent (94.8±0.1% (n=5) for day 1 and 94.3±0.5% (n=4) for day 2), as was the radioactivity recovery (101±3% (n=5) for day 1 and 102±2% (n=4) for day 2). Values greater than 100% are likely a result of slight geometry-related biases that occur in the dose calibrator, e.g., when measuring the activity of a vial versus a chip. Only ˜1% of radioactivity remained stuck to the chip (as unrecoverable activity) on both days.

Notably, the synthesis conditions were taken directly from previous manual efforts to optimize the synthesis of [¹⁸F]fallypride, with no need for re-optimization. The synthesis performance on the new automated system was very similar to manually-performed syntheses during the optimization studies (see Table 2 below). The similarity is not surprising considering that the high-throughput studies used similar microfluidic chips, but containing a 2×2 array of circular hydrophilic reaction sites (each 4 mm diameter). The fluorination efficiency of the two methods was the same (94.6±0.4% (n=9) for the automated chip, compared to 95±1% (n=6) for the manually-performed high-throughput experiments). However, the radioactivity recovery was higher for the automated setup (101±3% (n=9) versus 91±1% (n=6)). This was due to the improved automated collection process, which eliminated losses due to manual pipetting. Consequently, the crude RCY obtained with the microdroplet reactor was 96±3% (n=9), about ˜10% higher than that obtained previously with the high throughput reactor (87±1% (n=6)) (see, e.g., Rios, A., Wang, J., Chao, P. H., & van Dam, R. M. (2019). A novel multi-reaction microdroplet platform for rapid radiochemistry outimization. RSC Advances. 9(35). 20370-20374).

TABLE 2

| | Day 1 (N = 5) | Day 2 (N = 4) |
|---|---|---|
| Radioactivity recovery (%) | 101 ± 3 | 102 ± 2 |
| Fluorination efficiency (%) | 94.8 ± 0.1 | 94.3 ± 0.5 |
| Crude RCY (%) | 95 ± 3 | 97 ± 2 |
| Residual activity on chip (%) | 0.7 ± 0.4 | 0.8 ± 0.2 |

Table 2 shows the comparison of [¹⁸F]fallypride syntheses performed on different days. Synthesis time for all experiments was ˜17 min. All measurements are decay corrected. All values are average±standard deviation, computed from the indicated number of measurements on each day.

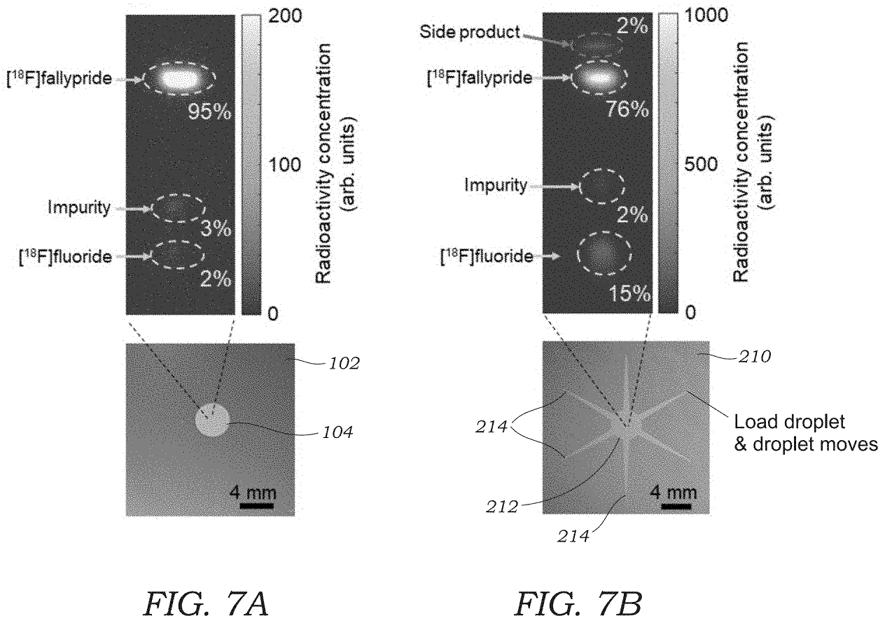

In contrast, the performance of the synthesis on our previous “passive transport” system was substantially lower, with crude RCY of 64±6% (n=4) (see J. Wang, P. H. Chao, S. Hanet and R. M. van Dam, Lab. Chip, 2017, 17, 4342-4355). However, this previous work was performed using different reaction conditions, making a meaningful comparison of the two technologies impossible. Therefore, the synthesis was performed on the passive transport chip using the same reaction conditions used in the current paper, and observed a crude RCY of 75±10% (n=5). This result suggests that the design improvements in the new droplet synthesis platform resulted in nearly 30% relative improvement in the RCY, i.e., from 75±10% (n=5) to 96±3% (n=9). By eliminating the hydrophilic reagent delivery “channels”, significant improvements were seen both in the fluorination efficiency as well as recovery efficiency. The increase in fluorination efficiency (i.e., from 81±9% (n=5) to 94.6±0.4% (n=9)) is due to better confinement of both the [¹⁸F]fluoride (during the drying step) and precursor (during the radiofluorination step) to the circular reaction site, leading to more uniform concentrations. On the previous passive transport chip, reagents were often slightly spread out along the passive “channels” (i.e., away from the reaction site), leading to unmixed regions and reduced amount of reagents at the actual reaction site. Example radio-TLC chromatograms (FIGS. 7A and 7B) confirm that the reaction on the passive transport chip has lower conversion and also has an extra radiolabeled side product. The amount of this side product was observed to increase when the radio of base to precursor increases, perhaps indicating that there are pockets of abnormally low or high concentrations of reagents during syntheses on the passive transport chip. The circular reaction site also helps to increase the radioactivity recovery (i.e., from 92±5% (n=5) to 101±3% (n=9)), presumably because all of the liquid remains confined to the central reaction region and can more efficiently be collected from the chip. For some experiments, Cerenkov imaging was performed to view the distribution of activity on the chip after collection of the crude product (FIG. 5). The residual activity on the circular reaction chip after collection was 0.7±0.3% (n=9) of the starting activity, and 90.6±5.6% (n=4) of the residual activity was retained within the reaction site (FIG. 5A). In contrast, the residual activity on the passive transport chip was significantly higher (7±1% (n=5) of the starting activity), and more than 93% of the residual activity was located on the reagent delivery channels (FIG. 5B) where it could not be recovered by the product collection mechanism. Interestingly, the amount of unrecoverable residual activity within the reaction site was similar for both chips (˜0.5% for the circular reaction chip vs ˜0.4% for the passive transport chip). Table 3 below shows a comparison of [¹⁸F]fallypride syntheses performed on the new automated droplet synthesis platform (circular reaction site), high-throughput chips (containing 2×2 array of circular reaction sites) and the previous automated passive transport reactor (single reaction site with six tapered droplet transport channels). The same reaction conditions were used in all cases. All measurements are decay corrected. All values are average±standard deviation, computed from the indicated number of measurements in each case.

TABLE 3

| | Automated operation | Manual operation | Passive transport |
|---|---|---|---|
| | on single-reaction chip | on high-throughput chip | reactor |
| Number of experiments | 9 | 6 | 5 |
| Radioactivity recovery (%) | 101 ± 3 | 91 ± 1 | 92 ± 5 |
| Fluorination efficiency (%) | 94.6 ± 0.4 | 95 ± 1 | 81 ± 9 |
| Crude RCY (%) | 96 ± 3 | 87 ± 1 | 75 ± 10 |
| Residual activity on chip (%) | 0.7 ± 0.3 | 0.12 ± 0.05 | 7 ± 1 |
| Residual activity on the reaction | 0.5 ± 0.3 (n = 4) | NA | 0.4 ± 0.2 |
| site (%) | | | |

By using this new chip design and corresponding apparatus, the crude RCY of [¹⁸F]fallypride synthesis was therefore meaningfully augmented.

In addition, the synthesis time was also slightly improved (˜17 min here compared to ˜20 min in previous work). The fast speed of the rotary actuator limited the amount of time needed to properly position the chip between steps, and the optimized collection procedure (with faster vacuum ramping speed) shaved a few minutes from the overall process time. Further synthesis time reduction may be possible by optimizing the position of dispensers and collection tube within a smaller angular range.

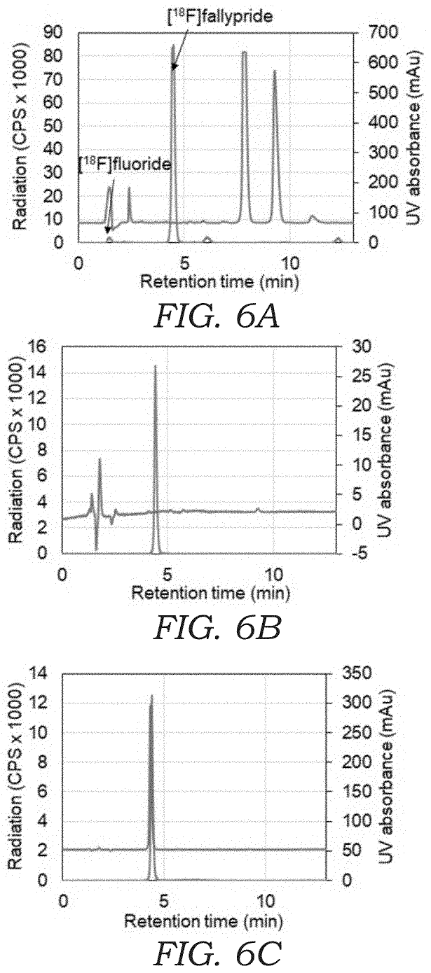

Though the main focus of this work was on developing a new chip and radiosynthesis system for improved and streamlined synthesis steps, we also performed purification of the crude product via analytical radio-HPLC. The purification efficiency was 81% (n=1) and overall RCY was 78% (n=1). Chromatograms of the crude product, purified product and purified product co-injected with fallypride reference standard are shown in FIG. 6. Due to the small amount of reagents (i.e., TBAHCO₃, precursor) used in microdroplet reactions, the crude product can be purified via analytical-scale HPLC compared to the semi-preparative HPLC used in conventional radiosynthesis. This results in short retention times (and short purification times) and lower mobile phase volume of the collected pure fraction (simplifying and shortening the formulation process). Furthermore, both the UV and radiation detector chromatograms of the crude [¹⁸F]fallypride product were in general much cleaner compared to the synthesis carried out in the macroscale (where overlap of product with impurities has been observed). In the radiation detector chromatogram, the product peak was sharp (˜0.5 min wide) and well separated from the [¹⁸F]fluoride peak and a couple of very small radioactive side-product peaks. In the UV chromatogram, the impurity peaks are well-defined and are well-separated from the product peak, making separation very straightforward. The needed purification time was only ˜5 min (retention time ˜4.5 min), and the purified product was 100% radiochemically pure.

A very compact (coffee cup-sized) microdroplet radiosynthesizer was developed for performing automated radiochemical reactions. The apparatus (10×6×12 cm, W×D×H) is over an order of magnitude smaller than commercial synthesizers that are currently considered to be very compact (e.g., IBA RadioPharma Solutions Synthera® has dimensions 17×29×28.5 cm, W×D×H). This could potentially allow much smaller shielding than a typical hot cell, or could allow a large number of synthesizers to be operated within a single hot cell.

Multi-step chemical reactions (including evaporative drying and radiofluorination) were performed to synthesize the PET tracer [¹⁸F]fallypride. The synthesis yield was very high and was consistent within a given day and from day to day. A significant advantage of this next-generation (rotary) platform compared to the previous passive transport approach is that the reaction site (hydrophilic circle) is identical to the shape of the reaction site on chips used for high-throughput reaction optimization (arrays of circular sites), eliminating the need for any reoptimization.

The small amount of reagents used in the microdroplet reactor resulted in a very clean chromatogram and short retention time (˜5 min) despite the purification being performed with only an analytical-scale HPLC column. The small volume of the mobile phase in the collected fraction (˜1.5 mL) could be rapidly removed via evaporation for reformulation in saline within ˜8 min. This time could potentially be further decreased using a microfluidic-based based PET tracer reformulation device.

Recently, the capability of producing [¹⁸F]fallypride on the passive transport chip at the GBq level by integrating the passive transport based reactor (see J. Wang, P. H. Chao, S. Hanet and R. M. van Dam, Lab. Chip, 2017, 17, 4342-4355) and a micro-cartridge based radionuclide concentrator. In that work, extensive studies were carried out to figure out how to optimally load ˜25 μL concentrated [¹⁸F]fluoride solution to the small reaction site without having the liquid spread out along the passive transport “channels” which can lead to poor mixing, low reaction efficiencies, and poor recovery of crude product. By integrating the concentrator with the presented next-generation microdroplet radiosynthesizer in the future, it will be much easier and faster to scale up the synthesis to clinically-relevant levels. FIG. 16 illustrates one such embodiment in which the microdroplet radiosynthesizer device is integrated with an upstream radioisotope concentrator and a downstream purification and/or formulation sub-system or module.

In addition to [¹⁸F]fallypride, this compact microdroplet reactor 100 can also be used for the synthesis of other PET tracers, such as [¹⁸F]FDOPA, [¹⁸F]FET, and [¹⁸F]Florbetaben ([¹⁸F]FBB), using substantially the same processes described with respect to FIGS. 4A-4C and FIGS. 12A-12B, with minor modifications and using reagents for the particular synthesis being performed. It has recently been shown that these other PET tracers can also be synthesized in high efficiency in droplet format, and can also be applied to labeling with other isotopes such as radiometals for both imaging and radiotherapeutic applications. Tools like Cerenkov imaging of chips will likely be helpful during the investigation of other tracers, for example to optimize reagent delivery parameters for new liquids (to prevent splashing of radioactivity outside the reaction site).

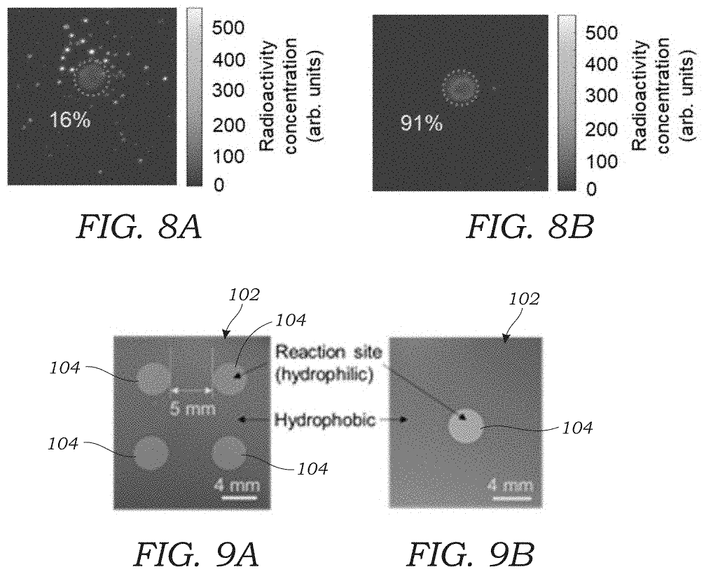

For example, during the preliminary study of using the microdroplet reactor to synthesize another tracer, [¹⁸F]FDOPA, we noticed signs of significant splashing of radioactivity outside of the reaction site (FIG. 8A) after observing the distribution of residual radioactivity (after the collection step) on a series of microfluidic chips via Cerenkov imaging. Suspecting that the addition of collection solution with the piezoelectric dispenser (driven at 69 kPa [10. psi]) may be causing some of the contents of the chip (crude product after fluorination reaction) to splash, we repeated experiments using a lower driving pressure (35 kPa [5.0 psi]) and observed that the signs of splashing disappeared (FIG. 8B). The initially high residual activity on the chip after collection (17%) was lowered to 5% with this change in the driving pressure. Since all other reagents are driven at 69 kPa [10. psi] without signs of splashing, this study indicated that delivery of each reagent (or solvent) involved in the synthesis may require a little bit of optimization, to determine the best dispensing pressure, as new tracers are explored.

[¹⁸F]FDOPA Synthesis

Here, a diaryliodonium salt based method of synthesizing [¹⁸F]FDOPA was implanted in a microdroplet format. We focused on this method due to the simple synthesis process and the commercial availability of the precursor. We optimized the synthesis protocol by testing various parameters, including concentrations of base and precursor, and reaction temperature. In addition, we investigated the use of the radical scavenger 2,2,6,6-tetramethyl-1-piperidinyloxy (TEMPO) to increase yield through prevention of precursor decomposition during the reaction. Furthermore, we automated the synthesis on the compact radiosynthesis device described herein.

The initial microscale [¹⁸F]FDOPA synthesis protocol was adapted from the macroscale synthesis method reported by Kuik et al. Experiments were first performed on multi-reaction microfluidic chips to optimize the protocol in a more high-throughput fashion, and then the synthesis with optimal conditions was automated. Optimization experiments were performed on microfluidic chips comprising a 2×2 arrays of circular hydrophilic reaction sites (4 mm diameter, 9 mm pitch (center-to-center spacing)) patterned in a hydrophobic substrate (25 mm×27.5 mm) (FIG. 9A). The patterned chips were prepared as described previously (except that no final acid treatment step was used) by coating silicon wafers with Teflon® AF, and then etching away the coating to leave exposed silicon regions. The microfluidic chip was affixed atop of a heater platform to control temperature, and reagent addition and crude product collection were performed with a micro-pipette. Each chip was used once and then discarded after use.

Reagents

Anhydrous acetonitrile (MeCN, 99.8%), methanol (MeOH, 99.9%), ethanol (EtOH, 99.5%), diethylene glycol dimethyl ether (diglyme, 99.8%), TEMPO (98%), potassium carbonate (K₂CO₃, 99%), 4,7,13,16,21,24-hexaoxa-1,10-diazabicyclo[8.8.8]hexacosane (K222, 98%), hydrocholoric acid (HCl, 37%), sulfuric acid (H₂SO₄, 99.99%), ethylenediaminetetraacetic acid (EDTA, 99%), acetic acid (99%), L-ascorbic acid and perchloric acid (HClO₄) were purchased from Sigma-Aldrich. Both 6-Fluoro-L-DOPA hydrochloride (reference standard for L type [¹⁸F]FDOPA) and 6-Fluoro-D,L-DOPA hydrochloride (reference standard for mixture of D and L type [¹⁸F]FDOPA) were purchased from ABX Advanced Biochemical Compounds (Radeberg, Germany). ALPDOPA precursor was obtained from Ground Fluor Pharmaceuticals (Lincoln, NB, USA). DI water was obtained from a Milli-Q water purification system (EMD Millipore Corporation, Berlin, Germany). No-carrier-added [¹⁸F]fluoride in [¹⁸O]H₂O was obtained from the UCLA Ahmanson Biomedical Cyclotron Facility.

Prior to synthesis of [¹⁸F]FDOPA, several stock solutions were prepared. Base stock solution was prepared by dissolving K₂₂₂ (22.8 mg) and K₂CO₃ (4.08 mg) in a 9:1 (v/v) mixture of DI water and MeCN (600 μL). [¹⁸F]fluoride stock solution (containing 8.4 mM K₂₂₂ and 4.1 mM K₂CO₃) was prepared by mixing [¹⁸F]fluoride/[¹⁸O]H₂O (10 μL, ˜220 MBq [˜6.0 mCi]), base solution (10 μL) and DI water (100 μL). Precursor stock solution (containing 9 mM ALDOPA) was prepared by dissolving ALDOPA (0.96 mg) in diglyme (120 μL, 75 mol % TEMPO). Finally, a collection solution to dilute the crude product prior to collection from the chip was prepared from a 4:1 (v/v) mixture of MeOH and DI water (500 μL).

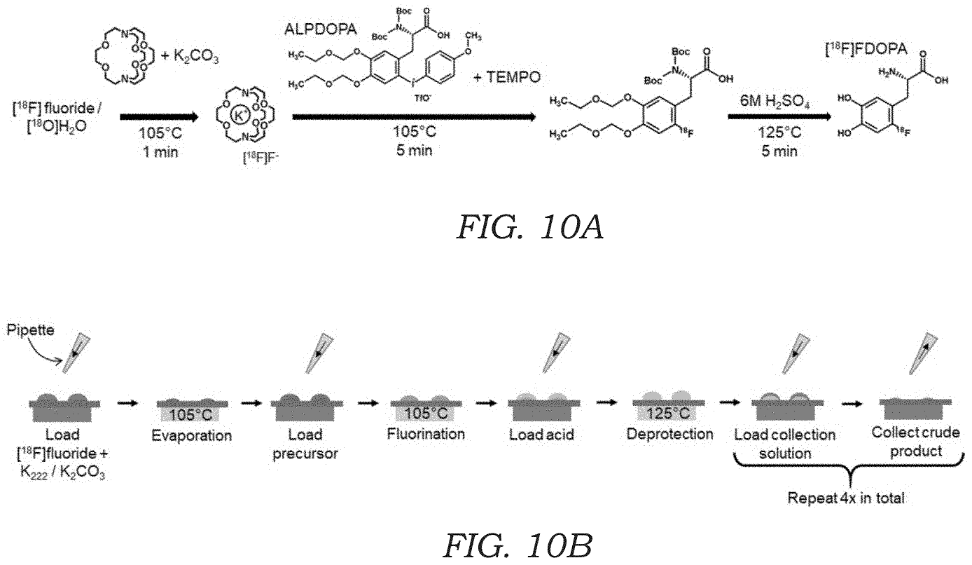

The details of the manual microscale synthesis are shown in FIG. 10B while FIG. 10A illustrates the synthesis scheme. Briefly, a 10 μL droplet of [¹⁸F]fluoride stock solution (˜11 MBq, 84 nmol K₂₂₂/41 nmol K₂CO₃) was first loaded on each reaction site, and the chip was heated to 105° C. for 1 min to form the dried [¹⁸F]KF/K₂₂₂ complex at each site. Then, a 104 droplet of precursor solution was added to reach reaction site and the chip was heated to 100° C. to perform the fluorination step. During the 5 min reaction, the solvent was replenished at all sites by adding droplets (˜7 μL) of diglyme every 30 s. Following fluorination, a 10 μL droplet of H₂SO₄ (6M) was added to each reaction site and the mixtures were heated to 125° C. for 5 min to perform the deprotection step. Finally, for each individual reaction site, a 20 μL droplet of collection solution was loaded at each site to dilute the resulting crude product, which was then recovered via pipette. The dilution and collection process was repeated 4× in total to maximize the radioactivity recovery.

Performance of the fluorination step was assessed through measurements of radioactivity using a calibrated dose calibrator (CRC-25R, Capintec, Florham Park, N.J., USA) at various stages of the synthesis process, and measurements of fluorination efficiency using radio thin-layer chromatography (radio-TLC). All radioactivity measurements were corrected for decay. Radioactivity recovery was calculated as the ratio of radioactivity of the collected crude product to the starting radioactivity on the chip after loading the [¹⁸F]fluoride stock solution. Residual activity on the chip was the ratio of radioactivity on the chip after collection to the starting radioactivity on the chip. Fluorination efficiency of the crude product collected from the chip was determined via radio-TLC as described below. Fluorination yield (decay-corrected) was defined as the radioactivity recovery times the fluorination efficiency.

To accelerate the analysis, radio-TLC was performed using recently-developed parallel analysis methods. Groups of 4 samples were spotted via pipette (1 μL each, 1 mm pitch) onto each TLC plate (silica gel 60 F₂₅₄ TLC plate, aluminum backing (Merck KGaA, Darmstadt, Germany)). TLC plates were dried in air and developed in the mobile phase (95:5 v/v MeCN:DI water). After separation, the multi-sample TLC plate was read out by imaging (5 min exposure) with a custom-made Cerenkov luminescence imaging (CLI) system. To determine the fluorination efficiency, regions of interest (ROIs) were drawn on the final image (after image corrections and background subtraction) to enclose the radioactive regions/spots. Each ROI was integrated, and then the fraction of the integrated signal in that ROI (divided by the sum of integrated signal in all ROIs) was computed. Two radioactive species were separated in the samples: [¹⁸F]fluoride (Rf=0.0) and the fluorinated intermediate (Rf=1.0).

Before developing our multi-reaction microfluidic chips, we performed some initial studies of the fluorination step with varied reaction conditions to establish a baseline set of conditions upon which further fine-grained optimizations could be made. The initial studies examined reaction temperature (85-125° C.), reaction time (5-15 min), reaction solvent (DMF, MeCN, DMSO, diglyme), precursor concentration (9-71 mM), base amount (21-168 nmol of K₂₂₂ and 10-82 nmol of K₂CO₃). The highest fluorination yield (˜7%) was observed using 84 nmol K₂₂₂/41 nmol K₂CO₃, 9 mM precursor, diglyme as reaction solvent, 105° C. temperature, and 5 min reaction time, but the yield exhibited poor day to day consistency.

Previously, Carroll et al. reported that the yield and reproducibility of the fluorination of diaryliodonium salts could be improved by adding TEMPO as a radical scavenger to improve the stability of the diaryliodonium salt precursor; we investigated whether this approach could be potentially used to improve the yield and consistency of [¹⁸F]FDOPA synthesis using the multi-reaction chips.

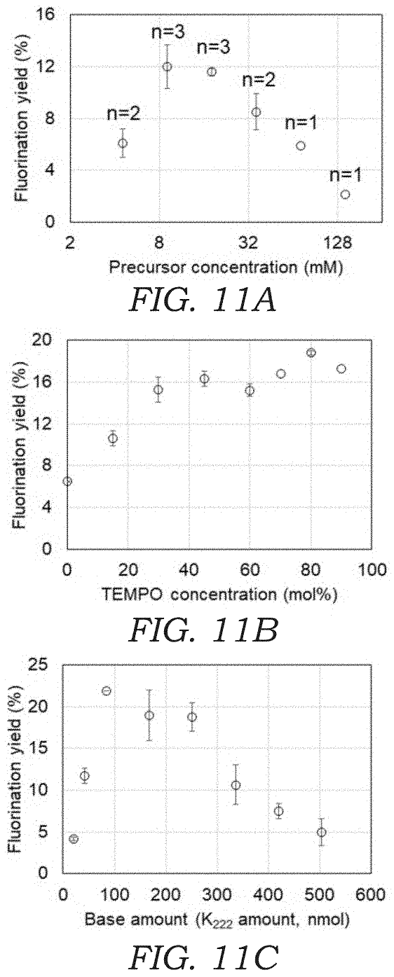

Initially we added 20 mol % TEMPO into the precursor solution, and performed a detailed study of the effect of precursor concentration on the fluorination yield (FIG. 11A) with 5 min reaction time and 105° C. reaction temperature. The highest yields were obtained with moderate precursor concentrations. At 9 mM and 18 mM, the fluorination yields were 12.0±1.7% (n=3) and 11.6±0.3% (n=3), respectively. We chose 12 mM for subsequent experiments to study of the effect of TEMPO concentration on the fluorination step (FIG. 11B). The fluorination yield was only 6.5±0.1% (n=2) without any TEMPO but nearly tripled (18.8±0.2% (n=2)) when 80 mol % TEMPO was added. The improvement was mainly due to an increase in fluorination efficiency from 23±1% (n=2) to 53±2% (n=2), respectively, though a small increase in radioactivity recovery (from 28±2% (n=2) to 35±2% (n=2), respectively) was also observed. Next, we studied the effect of the amount of base, keeping the ratio of K₂₂₂ at K₂CO₃ fixed at 2.05. (FIG. 11C). As the amount of base was increased, starting from 21 nmol K₂₂₂/10 nmol K₂CO₃, the fluorination yield rose sharply and reached the maximum, 21.89±0.02% (n=2) at 84 nmol K₂₂₂/41 nmol K₂CO₃). The fluorination yield remained relatively constant up to −252 nmol K₂₂₂/123 nmol K₂CO₃ (18.8±1.7% (n=2)), and then began to drop significantly as base amount was further increased. Thus, for the later deprotection study, we picked 75 mol % TEMPO, 9 mM precursor solution, 84 nmol K₂₂₂/41 nmol K₂CO₃ as base amount.

Deprotection was performed immediately after fluorination, with no intermediate purification step. To assess the performance of this step, the [¹⁸F]FDOPA conversion after deprotection was assessed via radio high-performance liquid chromatography (HPLC) as described below. Crude radiochemical yield (RCY, decay-corrected) was defined as the radioactivity recovery times the [¹⁸F]FDOPA conversion. Isolated RCY was defined as the ratio of radioactivity of the purified product (recovered from the same analytical-scale radio-HPLC) to the starting radioactivity on the chip.

Analysis of samples (crude reaction mixture or purified product) was performed on a Smartline HPLC system (Knauer, Berlin, Germany) equipped with a degasser (Model 5050), pump (Model 1000), a UV detector (Eckert & Ziegler, Berlin, Germany) and a gamma-radiation detector and counter (B-FC-4100 and BFC-1000; Bioscan, Inc., Poway, Calif., USA). Injected samples were separated with a C18 column (Luna, 5 μm pore size, 250×4.6 mm, Phenomenex, Torrance, Calif., USA). The mobile phase consisted of 1 mM EDTA, 50 mM acetic acid, 0.57 mM L-ascorbic acid and 1% v/v EtOH in DI water. The flow rate was 1.5 mL/min and UV absorbance detection was performed at 280 nm. The retention times of [¹⁸F]fluoride, [¹⁸F]FDOPA and the fluorinated intermediate were 2.4, 6.2, and 25.8 min, respectively. [¹⁸F]FDOPA conversion was determined via dividing the area under the [¹⁸F]FDOPA peak by the sum of areas under all three peaks.

For purification, the collected crude product (˜80 μL) was first diluted with 80 μL of the mobile phase, and then separated under the same conditions as above.

For some experiments, The enantiomeric purity was verified by co-injecting the purified product and mixture of D and L type reference standard and separated using a chiral column (Crownpack CR(+), 5 μm, 150×4 mm, Chiral Technologies, West Chester, Pa., USA) using a mobile phase of HClO₄ solution (pH=2) at a flow rate of 0.8 mL/min. Retention times of L-DOPA and D-DOPA were 9.5 and 12.1 min, respectively.

Preliminary optimization of the deprotection step (deprotection reagent, concentration, reaction temperature and reaction time) is summarized in the Table 4 below. Using single-reaction microfluidic chips, the influence of several deprotection reaction parameters was investigated, including type of acid (HCl and H₂SO₄), acid concentration, reaction time, and reaction temperature. These experiments were performed prior to complete optimization of the fluorination step, and used 84 nmol K₂₂₂, 41 nmol K₂CO₃, 36 mM precursor, and 20 mol % TEMPO.

TABLE 4

| Deprotection reagent | HCl | | | | H₂SO₄ | | | |
|---|---|---|---|---|---|---|---|---|
| Concentration (M) | 6 | | | | 3 | 6 | | |
| Deprotection time (min) | 5 | 10 | 15 | 15 | 5 | 5 | | |
| Deprotection temperature (° C.) | 90 | 90 | 90 | 100 | 100 | 120* | 130 | 140 |
| Radioactivity loss (%) | 86 | 88 | 86 | 88 | 78 | 84 ± 3 | 90 | 87 |
| Residual activity on chip (%) | 3 | 1 | 2 | 1 | 3 | 3 ± 1 | 2 | 2 |
| Radioactivity recovery (%) | 8 | 8 | 10 | 8 | 15 | 9 ± 1 | 6 | 7 |
| [¹⁸F]FDOPA conversion (%) | 24 | 37 | 53 | 72 | 42 | 87 ± 1 | 83 | 92 |
| Crude RCY (%) | 2.0 | 3.1 | 5.2 | 5.5 | 6.3 | 7.2 ± 0.5 | 4.9 | 6.8 |
| Isolated RCY (%) | 1.4 | 2.7 | 4.0 | 4.5 | 4.5 | 4.8 ± 0.6 | 3.2 | 3.7 |

Table 4 shows the effect of various deprotection conditions (without cover plate). Radioactivity loss indicates the combined activity losses (due to formation of volatile species) during evaporation, fluorination and deprotection steps. Percentages are corrected for decay. For most conditions, only n=1 experiment was performed. * indicates n=2 replicates were performed, and values indicate average±standard deviation.

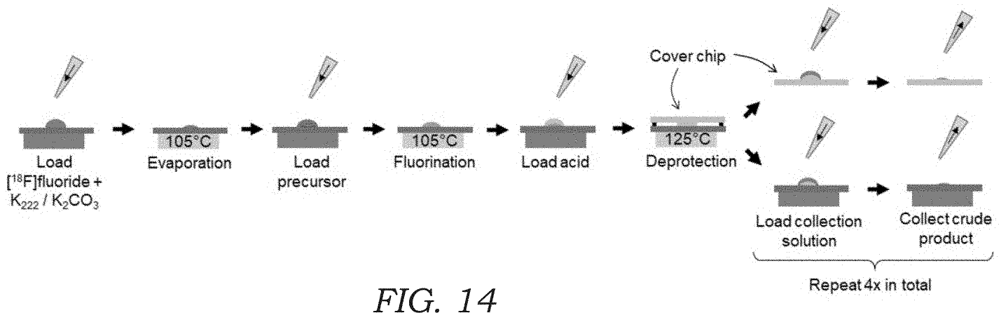

Even though the overall crude RCY and isolated RCY were below 10% due to performing these experiments starting with non-optimal fluorination conditions (i.e., 20 mol % TEMPO, 36 mM precursor, 84 nmol K₂₂₂/41 nmol K₂CO₃), comparative conclusions could still be drawn. Performing deprotection with 6 M H₂SO₄ at 115° C. enabled the highest RCY. Combining these conditions with the optimal fluorination conditions, [¹⁸F]FDOPA could be produced on the chip with crude RCY of 11% (n=1) and isolated RCY of 7.2% (n=1). By adding a cover plate over the droplet during deprotection (FIG. 14 and Table 5), the crude RCY and isolated RCY could be further increased to 14.3±0.5% (n=2) and 10.0±0.7% (n=2), respectively. Noting that the [¹⁸F]FDOPA conversion was only 84±5% (n=2) at 115° C., indicating the deprotection reaction was not complete, we increased the deprotection temperature to 125° C. and the conversion improved to 95% (n=1).

TABLE 5

| | No cover plate | With cover plate |
|---|---|---|
| | (n = 1) | (n = 2) |
| Radioactivity loss (%) | 84 | 53.7 ± 0.4 |
| Residual activity on cover chip (%) | NA | 26 ± 2 |
| Residual activity on bottom chip (%) | 3 | 1.5 ± 0.2 |
| Radioactivity recovery (%) | 12 | 17 ± 2 |
| [¹⁸F]FDOPA conversion (%) | 91 | 84 ± 5 |
| Crude RCY (%) | 11.0 | 14.3 ± 0.5 |
| Isolated RCY (%) | 7.2 | 10.0 ± 0.7 |

Table 5 shows the effect of cover plate on the synthesis performance. Radioactivity loss indicates the combined activity losses (due to formation of volatile species) during evaporation, fluorination and deprotection steps. Percentages are corrected for decay. Values of the group with cover plate indicate average±standard deviation computed from the indicated number of replicates.

Finally, we performed full (manual) syntheses including analytical-scale HPLC purification and formulation. The fluorination conditions were 75 mol % TEMPO, 9 mM precursor solution, 84 nmol K₂₂₂/41 nmol K₂CO₃ at 105° C. for 5 min, and the deprotection conditions were 6M H₂SO₄ at 125° C. for 5 min (with cover plate). The resulting crude RCY and isolated RCY were 20.5±3.5% (n=3) and 15.1±1.6% (n=3), respectively (Table 6 below).

TABLE 6

| | Manual | Automated |
|---|---|---|
| | synthesis | synthesis |
| | (n = 3) | (n = 3) |
| Starting activity (MBq) | 4.4~12.2 | 12.6~22.9 |
| Synthesis time including purification (min) | ~40 | ~37 |
| [¹⁸F]FDOPA conversion (%) | 95.6 ± 0.4 | 78 ± 4 |
| Crude RCY (%) | 20.5 ± 3.5 | 15.2 ± 2.1 |
| Isolated RCY (%) | 15.1 ± 1.6 | 10.3 ± 1.4 |
| Enantiomeric purity (%) | 98.0 ± 0.2 | N.M. |
| Total activity loss during overall synthesis (%) | 50 ± 5 | 78 ± 2 |
| Unrecoverable activity on cover chip (%) | 24.7 ± 0.3 | NA |
| Unrecoverable activity on bottom chip (%) | 2.1 ± 0.4 | 2.9 ± 0.2 |
| Radioactivity recovery (%) | 21 ± 4 | 20 ± 2 |

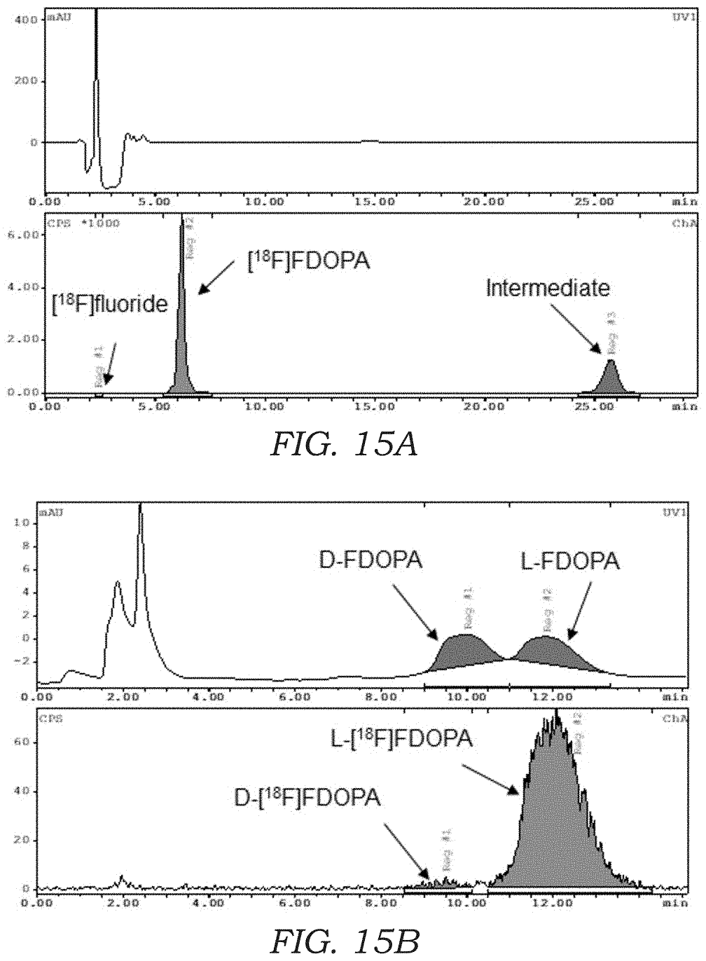

An example of a radio-HPLC chromatogram of the crude product is shown in FIG. 15A, and a co-injection with L-DOPA and D-DOPA reference standards to determine enantiomeric purity (98.0±0.2 (n=3)) is shown in FIG. 15B. The retention time of [¹⁸F]FDOPA was ˜6 min, and the chromatogram was relatively clean with no nearby side-product peaks, despite omission of the intermediate cartridge purification between fluorination and deprotection steps. The overall synthesis time was only ˜40 min, including ˜25 min for initial drying of [¹⁸F]fluoride and the two reactions, ˜7 min for purification and ˜8 min for formulation.

To increase safety and to facilitate routine production, we next automated the synthesis. Automated syntheses were conducted on chips with a single reaction site (FIG. 9B) operated using the compact radiosynthesis device described herein (FIGS. 2A and 2B), consisting of a rotating, temperature-controlled platform, a set of reagent dispensers, and a collection system to remove the reaction droplet at the end of the synthesis. The rotating stage positions the reaction site as desired under a carousel in which reagent dispensers and product collection tube are mounted.

Prior to synthesis, reagent vials connected to the reagent dispensers were loaded with the [¹⁸F]fluoride stock solution, precursor stock solution, replenishing solution (diglyme), deprotection solution (6M H₂SO₄) and collection solution. An illustration of the automated microdroplet radiosynthesis is shown in FIG. 12. The chip was first rotated to position the reaction site below the dispenser 1 for [¹⁸F]fluoride stock solution and ten 1 μL droplets of [¹⁸F]fluoride stock solution (˜18.5 MBq; ˜0.5 mCi) were sequentially loaded onto the chip (total time <10 s). The chip was rotated 45° counterclockwise (CCW) and heated to 105° C. for 1 min to evaporate the solvent and leave a dried residue of the [¹⁸F]KF/K₂₂₂ complex at the reaction site. Then, the chip was rotated 45° CCW to position the reaction site under the precursor dispenser and ten 1 μL droplets of precursor solution were loaded to dissolve the dried residue. Next, the chip was rotated 45° CCW to position the reaction site under the replenishing dispenser (diglyme) and heated to 100° C. for 5 min to perform the fluorination reaction. Solvent was replenished by adding a 1 μL droplet of diglyme every 10 s. Afterwards, the chip was rotated 45° CCW to position the reaction site under the deprotection solution dispenser, twenty 0.5 μL droplets of deprotection solution were loaded on the reaction site and the chip was heated to 125° C. for 5 min to perform deprotection step. Finally, the chip was rotated 45° CCW to position the reaction site under the collection solution dispenser, and twenty 1 μL droplets of collection solution were deposited to dilute the crude product. After rotating the chip 45° CCW to position the reaction site under the collection tube, the diluted solution was transferred into the collection vial by applying vacuum. The collection process was repeated a total of four times to minimize the residue on the chip (i.e., by rotating the chip 45° CW back to the collection solution dispenser, loading more collection solution, etc.).

Considering the accuracy of droplet volume dispensed by the dispensers (˜10%) studied previously, we adjusted some concentrations so the overall synthesis would be more robust and repeatable, and tolerant of volume errors. The optimal condition was selected where the slope of the optimization curves (in FIGS. 11A-11C) was close to zero. Automated syntheses were performed with 80 mol % TEMPO, 12 mM precursor solution and 101 nmol K₂₂₂/49 nmol K₂CO₃.

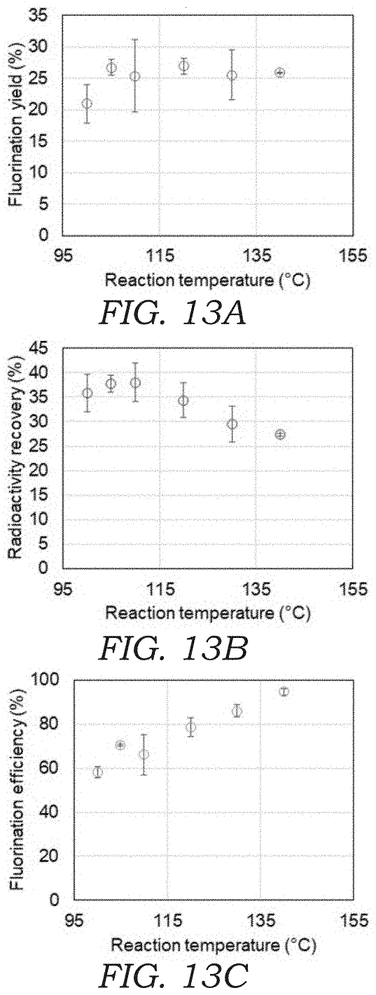

Benefiting from the automated dispensing system, the frequency of replenishing solvent during heated reactions could be increased (up to several droplets per second, compared to one droplet per ˜7 s via manual dispensing), and we therefore briefly explored higher fluorination temperatures. As shown in FIGS. 13A-13C, with the increase of reaction temperature from 100° C. to 140° C., even though the fluorination efficiency increases from 58±3% (n=3) to 95±1% (n=2), the radioactivity recovery fell from 36±4% (n=3) to 27.3±0.3% (n=2). Due to these opposite effects, the overall fluorination yield was relatively constant (˜26%) for temperatures above 105° C. Overall, 120° C. reaction temperature resulted in the highest fluorination yield of 26.9±1.3% (n=2) and was chosen as the optimal reaction temperature for the automated synthesis. As shown in Table 7, with full automated synthesis, the crude RCY and isolated RCY were 15.2±2.1% (n=3) and 10.3±1.4% (n=3), respectively.

TABLE 7

| | Manual | Automated |
|---|---|---|
| | synthesis | synthesis |
| | (n = 3) | (n = 3) |
| Starting activity (MBq) | 4.4~12.2 | 12.6~22.9 |
| Synthesis time including purification (min) | ~40 | ~37 |
| [¹⁸F]FDOPA conversion (%) | 95.6 ± 0.4 | 78 ± 4 |
| Crude RCY (%) | 20.5 ± 3.5 | 15.2 ± 2.1 |
| Isolated RCY (%) | 15.1 ± 1.6 | 10.3 ± 1.4 |
| Enantiomeric purity (%) | 98.0 ± 0.2 | N.M. |
| Total activity loss during overall synthesis (%) | 50 ± 5 | 78 ± 2 |
| Unrecoverable activity on cover chip (%) | 24.7 ± 0.3 | NA |
| Unrecoverable activity on bottom chip (%) | 2.1 ± 0.4 | 2.9 ± 0.2 |
| Radioactivity recovery (%) | 21 ± 4 | 20 ± 2 |

Both are slightly lower than the manual synthesis, which is commonly occurs when transferring from manual to automated synthesis protocol. We note that the [¹⁸F]FDOPA conversion was lower for the automated synthesis (i.e., 78±4% (n=3) vs 95.6±0.4% (n=3), respectively), likely due to the absence of the cover plate, which was omitted to avoid the need for manual intervention during operation, while the radioactivity recoveries of both methods were comparable (20±2% (n=3) vs 21±4% (n=3), respectively). To further increase the [¹⁸F]FDOPA conversion, we attempted performing the deprotection step at even higher temperature (130° C.), but significant side products appeared. The synthesis time was ˜22 min, which was slightly faster than the manual synthesis (˜25 min) due to the automation steps.

Compared to macroscale methods for [¹⁸F]FDOPA synthesis using the same precursor and route, the microscale method, with 10 μL reaction volume, used significantly less precursor, i.e., 0.12 μmol versus 16.8 μmol or 13.4 μmol. The small mass of reagents and small volume collected from the chip (˜80 μL) furthermore facilitated the use of analytical-scale HPLC to perform purification. This enabled rapid purification (˜7 min) and also needed only a short time for formulation (˜8 min). Overall the synthesis time with the microdroplet reactor was ˜37 min, compared to −71 min, or −117 min in conventional radiosynthesizers. In fact, the isolated non-decay-corrected yield of the microscale method 8.2±1.1% (n=3) (was higher than both macroscale approaches, i.e., 2.9±0.8% (n=3) and 6.7%±1.9% (n not reported).

Other than production of radiopharmaceuticals for imaging or therapy, our automated platform also has the potential to be applied for small scale chemical reactions or assays, in applications where compact apparatus and/or small reagent volumes are critical.

While embodiments of the present invention have been shown and described, various modifications may be made without departing from the scope of the present invention. For example, while the radiosynthesis device has been described largely in the context of moving the microfluidic chip relative to stationary dispensers and the collection it may be possible to reverse this configuration whereby the microfluidic chip is stationary while the dispensers and collection tube are moved by the motorized rotation stage. The invention, therefore, should not be limited, except to the following claims, and their equivalents.

1. A radiosynthesis device comprising:
a thermally controlled support configured to hold a microfluidic chip having one or more reaction sites formed thereon;
a fixture configured to hold a plurality of dispensers and a collection tube;
a plurality of non-contact dispensers installed on the fixture above the support and configured to respectively dispense one or more droplets of a respective reagent into the one or more reaction sites;
a collection tube installed on the fixture above the support; and
a motorized rotation stage operatively coupled to the support for controllably rotating the support, the motorized rotation stage configured to controllably rotate the support relative to the non-contact dispensers to sequentially position the one or more reaction sites for dispensing respective reagent from the non-contact dispensers into the one or more reaction sites, and to controllably rotate the support relative to the collection tube to sequentially position the one or more reaction sites for removing reaction product from the one or more reaction sites via the collection tube.

2. The radiosynthesis device of claim 1, further comprising a computing device having software executed thereon and configured to control a temperature of the thermally controlled support, the motorized rotation stage, dispensing of reagents by the non-contact dispensers and removal of reaction product by the collection tube.

3. The radiosynthesis device of claim 1, wherein the thermally controlled support comprises a heater and a thermoelectric cooler.

4. The radiosynthesis device of claim 3, further comprising a heat sink in thermal contact with one or more of the heater and the thermoelectric cooler.

5. The radiosynthesis device of claim 4, further comprising a fan coupled to the fixture and configured to move air over the heat sink.

6. The radiosynthesis device of claim 1, further comprising a collection vial fluidically coupled to the collection tube and respective reagent tubes fluidically coupled to the plurality of non-contact dispensers and to respective reagent containers coupled to the fixture.

7. The radiosynthesis device of claim 1, wherein the microfluidic chip comprises a plurality of hydrophilic reaction sites formed thereon and disposed along an arc on a surface of the microfluidic chip.

8. The radiosynthesis device of claim 2, further comprising a data acquisition device interfacing the computing device with the thermally controlled support, the motorized rotation stage, the non-contact dispensers, and the collection tube.

9. The radiosynthesis device of claim 1, wherein the motorized rotation stage and fixture are mounted within a housing which prevents the emission of materials and provides radiation shielding.

10. The radiosynthesis device of claim 1, wherein the radiosynthesis device has a size less than about 750 cm³.

11. The radiosynthesis device of claim 1, wherein the one or more of the plurality of non-contact dispensers, reagent vials, reagent tubing, and the collection tube are disposed in a cartridge that is removably mounted to the fixture.

12. The radiosynthesis device of claim 1, wherein the support comprises on one or more positioning elements for accurately positioning and securing the microfluidic chip on the thermally controlled support.

13. A radiosynthesis device comprising:
a thermally controlled support configured to hold a microfluidic chip having one or more reaction sites formed thereon, wherein the support maintains the microfluidic chip stationary; and
a motorized rotation stage; and
a plurality of non-contact dispensers and a collection tube operatively coupled to the motorized rotation stage and disposed above the microfluidic chip;
wherein the motorized rotation stage is configured to controllably rotate the non-contact dispensers and a collection tube relative to the support to sequentially position the non-contact dispensers and a collection tube at the one or more reaction sites.

14. A radiosynthesis system comprising:
a radioisotope concentrator configured to concentrate a radioisotope and output the radioisotope to the radiosynthesis device of claim 1; and
a downstream purification and/or formulation module configured to receive a radiochemical compound synthesized by the radiosynthesis device.

15. The radiosynthesis system of claim 14, further comprising a downstream formulation module configured to receive a radiochemical compound synthesized by the radiosynthesis device.

16. A method of using the radiosynthesis device of claim 1, comprising:
dispensing one or more droplets of reagent onto the one or more reaction sites of the microfluidic chip using the plurality of non-contact dispensers, wherein the microfluidic chip is rotated into position under respective non-contact dispensers by the motorized rotation stage;
heating and/or cooling the one or more droplets of reagent using the thermally controlled support;
rotating the microfluidic chip to place the one or more reaction sites containing a droplet thereon under the collection tube; and
removing reaction product with the collection tube by applying a vacuum to the collection tube.

17. A method of using the radiosynthesis device of claim 1 to produce a radiochemical, comprising:
dispensing one or more droplets of a radioisotope stock solution comprising a radioisotope in a solvent onto a first reaction site of the one or more reaction sites of the microfluidic chip using a first dispenser of the plurality of non-contact dispensers;
thermally treating the radioisotope stock solution on the first reaction site using the thermally controlled support to evaporate the solvent leaving a dried residue of radioisotope complex on the first reaction site;
rotating the microfluidic chip by rotating the motorized rotation stage to position the first reaction site at a second dispenser of the plurality of non-contact dispensers;
dispensing one or more droplets of a precursor solution onto the first reaction site using the second dispenser to dissolve the dried residue of radioisotope complex resulting in a solution of precursor solution and radioisotope complex;
rotating the microfluidic chip by rotating the motorized rotation stage to position the first reaction site at a third dispenser of the plurality of non-contact dispensers;
with the first reaction site positioned at the third dispenser, thermally treating the solution of precursor solution and radioisotope complex on the first reaction site using the thermally controlled support to perform a radiofluorination reaction and periodically dispensing a replenishing reagent onto the first reaction site using the third dispenser during the radiofluorination reaction, thereby producing a fluorinated reaction product;
rotating the microfluidic chip by rotating the motorized rotation stage to position the first reaction site at a fourth dispenser of the plurality of non-contact dispensers;
dispensing one or more droplets of a deprotection solution onto the first reaction site containing the fluorinated reaction product using the fourth dispenser;
thermally treating the deprotection solution and fluorinated reaction product on the first reaction site using the thermally controlled support to perform a deprotection reaction thereby producing crude radiochemical product;
rotating the microfluidic chip by rotating the motorized rotation stage to position the first reaction site at a fifth dispenser of the plurality of non-contact dispensers;
dispensing one or more droplets of a collection solution onto the first reaction site containing crude radiochemical product to dilute the crude radiochemical product using the fifth dispenser;
rotating the microfluidic chip by rotating the motorized rotation stage to position the first reaction site at the collection tube;
removing the diluted crude radiochemical product using the collection tube by applying a vacuum to the collection tube.

18. The method of claim 17, wherein the step of removing the diluted crude radiochemical product with the collection tube by applying a vacuum to the collection tube, comprises:
repeating the following collection process multiple times:
rotating the microfluidic chip by rotating the motorized rotation stage to position the first reaction site back to the fifth dispenser and dispensing one or more droplets of a collection solution onto the first reaction site containing crude radiochemical product; and
rotating the microfluidic chip by rotating the motorized rotation stage to position the first reaction site at the collection tube and removing the diluted crude radiochemical product with the collection tube by applying a vacuum to the collection tube.

19. The method of claim 18, wherein the collection process is repeated at least 3 times.

20. (canceled)

21. A method of using the radiosynthesis device of claim 1 to produce a radiochemical, comprising:
dispensing one or more droplets of a radioisotope stock solution onto a first reaction site of the one or more reaction sites of the microfluidic chip using a first dispenser of the plurality of non-contact dispensers;
rotating the microfluidic chip by rotating the motorized rotation stage to position the first reaction site at a second dispenser of the plurality of non-contact dispensers;
dispensing one or more droplets of a first reagent onto the first reaction site using the second dispenser resulting in a first reaction solution;
heating the first reaction solution using the using the thermally controlled support thereby producing a first reaction product;
cooling the first reaction product using the using the thermally controlled support;
rotating the microfluidic chip by rotating the motorized rotation stage to position the first reaction site at the collection tube;
removing radiochemical product in the first reaction site using the collection tube by applying a vacuum to the collection tube.

22. The method of claim 21, further comprising:
after the step of cooling the first reaction, and prior to removing the material in the first reaction site, performing the following steps:
rotating the microfluidic chip by rotating the motorized rotation stage to position the first reaction site at a third dispenser of the plurality of non-contact dispensers;
dispensing one or more droplets of a second reagent onto the first reaction site using the third dispenser resulting in a second reaction solution;
heating the second reaction solution using the using the thermally controlled support thereby producing a second reaction product; and
cooling the second reaction product using the using the thermally controlled support.

23. The method of claim 22, further comprising:
after the step of cooling the second reaction product, and prior to removing the material in the first reaction site, performing the following steps:
rotating the microfluidic chip by rotating the motorized rotation stage to position the first reaction site at a fourth dispenser of the plurality of non-contact dispensers;
dispensing one or more droplets of a third reagent onto the first reaction site using the third dispenser resulting in a third reaction solution;
heating the third reaction solution using the using the thermally controlled support thereby producing a third reaction product;
cooling the third reaction product using the using the thermally controlled support.

24-25. (canceled)
